SEQUENCE LISTING (8 sequences)

SEQ ID NO: 1 - DNA, 18 bp, Artificial Sequence
  Description of Artificial Sequence Synthetic linker sequence
agttcacgatccaagctg

SEQ ID NO: 2 - DNA, 18 bp, Artificial Sequence
  Description of Artificial Sequence Synthetic linker sequence
atcctgaagccacgagtt

SEQ ID NO: 3 - RNA, 17 nt, Artificial Sequence
  Description of Artificial Sequence Synthetic linker sequence
gaugguuacuccaagcg

SEQ ID NO: 4 - DNA, 20 bp, Artificial Sequence
  Description of Artificial Sequence Synthetic primer
taaccccgccccgttgacgc

SEQ ID NO: 5 - DNA, 24 bp, Artificial Sequence
  Description of Artificial Sequence Synthetic primer
ggcctgttcttccacctccattct

SEQ ID NO: 6 - DNA, 60 bp, Artificial Sequence
  Description of Artificial Sequence Synthetic primer
cccatatggatccagctaggcgcagatctttaacaccatgggggtgcccgaacgtcccac

SEQ ID NO: 7 - DNA, 60 bp, Artificial Sequence
  Description of Artificial Sequence Synthetic primer
gtgggacgttcgggcacccccatggtgttaaagatctgcgcctagctggatccatatggg

SEQ ID NO: 8 - protein, 6 aa, Artificial Sequence
  Description of Artificial Sequence Synthetic 6xHis tag
HHHHHH

(19) United States
(12) Patent Application Publication
(10) Pub. No.: US 2005/0260164 A1
(43) Pub. Date: Nov. 24, 2005

(54) Gene regulation with aptamer and modulator complexes for gene therapy

(76) Inventors: Doug Jolly, Oxford (GB); Jonathan Rohll, Oxford (GB); Bernd Buehler, Oxford (GB); Kyri Mitrophanous, Oxford (GB); Pippa Radcliffe, Oxford (GB)

Correspondence Address: FROMMER LAWRENCE & HAUG, 745 FIFTH AVENUE- 10TH FL., NEW YORK, NY, 10151, US

(21) Appl. No.: 11/102,428

(22) Filed: Apr. 8, 2005

Related U.S. Application Data
(63) Continuation-in-part of application No. PCT/US03/32035, filed on Oct. 9, 2003.
(60) Provisional application No. 60/417,456, filed on Oct. 10, 2002.

(51) Int. Cl. C12Q 1/68; A61K 48/00; C12N 15/867

(52) U.S. Cl. 424/93.2; 435/456

(57) ABSTRACT

Provided is an improved method for controlling gene expression in vivo through the use of a gene switch comprising one or more aptamer sequences operably linked to or incorporated into the untranslated regions (UTRs) of a transgene or nucleotide sequence of interest. Also provided are expression vectors having aptamer sequences located within the 3′ UTR, the 5′ UTR and between the genes of a multicistronic mRNA, as well as methods of using the expression vectors for controlling or regulating gene expression.

### CROSS-REFERENCE TO RELATED APPLICATIONS

This application is a continuation-in-part of International Application No. PCT/US2003/032035, filed on Oct. 9, 2003, published as WO 2004/033653 on Apr. 22, 2004, and claiming priority to U.S. application Ser. No.60/417,456 filed on Oct. 10, 2002.

Reference is made to U.S. application Ser. No. 10/008,610, filed Nov. 8, 2001; International application no. PCT/GB01/04433, filed Oct. 5, 2001 and published Apr. 11, 2002; International application no. PCT/GB02/05901, filed Dec. 23, 2002 and published Jul. 10, 2003; UK application Serial No. GB 0130797.4, filed Dec. 21, 2001; UK application Serial No. GB 0201140.1, filed Jan. 18, 2002; UK application Serial No. GB 0211409.8, filed May 17, 2002; U.S. application Ser. No. 10/082,122, filed Feb. 26, 2002; and U.S. application Ser. No.10/421,947, filed Apr. 24, 2003.

Each of the foregoing applications and patents, and each document cited or referenced in each of the foregoing applications and patents, including during the prosecution of each of the foregoing applications and patents (“application cited documents”), and any manufacturer's instructions or catalogues for any products cited or mentioned in each of the foregoing applications and patents and in any of the application cited documents, are hereby incorporated herein by reference. Furthermore, all documents cited in this text, and all documents cited or referenced in documents cited in this text, and any manufacturer's instructions or catalogues for any products cited or mentioned in this text or in any document hereby incorporated into this text, are hereby incorporated herein by reference. Documents incorporated by reference into this text or any teachings therein may be used in the practice of this invention. Documents incorporated by reference into this text are not admitted to be prior art. Furthermore, authors or inventors on documents incorporated by reference into this text are not to be considered to be “another” or “others” as to the present inventive entity and vice versa, especially where one or more authors or inventors on documents incorporated by reference into this text are an inventor or inventors named in the present inventive entity.

### FIELD OF THE INVENTION

This invention relates to methods for controlling recombinant gene expression in vivo by the use of aptamers complexed with modulator compounds.

### BACKGROUND OF THE INVENTION

Aptamers are short oligonucleotides which form specific structures in the presence of compounds, including small molecules. They are typically 20 to 200 base pairs in length and can be rapidly selected in vitro from a random library for high-affinity binding to such compounds. Aptamers have been used to control gene expression in a variety of settings, in target validation studies (Werstuck and is Green, Science, 282-296-8 (1998)); in diagnostic applications (Jayasena, Clinical Chemistry 45:9, 1628-1650 (1999)); in biosensors (Potyrailo et al., Anal. Chem. 70:3419-3425 (1998); and in treatment of solid tumors (U.S. patent application No.2002/0034506, entitled “Method for Treatment of Tumors Using Nucleic Acid Ligands to PDGF”). The technique by which the aptamers are obtained is called the systematic evolution of ligands by exponential enrichment (“SELEX”) process and is described in detail in Tuerk et al., Science 249:505-510 (1990) (FIG. 1). These methods use aptamers supplied externally to interact with various protein factors. In addition, it has been shown that a single copy of an RNA aptamer, three copies of the same aptamer, or a combination of two different aptamers, when incorporated into the 5′ untranslated region of a transgene can regulate gene expression at the translational level (Werstuck and Green op cit.; U.S. patent Publication 2002/0006661, filed Apr. 2, 2001; U.S. patent Publication 2003/0036173, filed Sep. 26, 2002). This action may be through secondary structure changes in the resulting mRNA in the presense of certain compounds or by other means such as ribosome blockade. This approach used antibiotics such as kanaymcin, tobramycin or Hoechst dyes as compounds that bind to aptamers. In principle this seems like an attractive method to control expression of genes that have been inserted into living cells, but a number of issues remain to be solved before this approach can have real utility. Firstly, these compounds have limited, if any clinical use because they are highly toxic or have no history of human use. Secondly the affinities observed were relatively low and not in the range that would lead to an expectation of efficient action in a whole animal or patient, given the concentrations of drugs that can normally be achieved in the bloodstream.

Another means to control gene expression is with an exogenous signal (“gene-switch”) that binds with high-affinity binding to a protein complex. There have been numerous methods proposed to achieve this regulation (See Gossen, M. et al., Science 268:1766-1769 (1995); Rivera, et al., Nature Medicine 2:1028-1032 (1996); Abruzzese et al., Hum. Gene Ther. 10:1499-1507 (1999); No et al., Proc. Natl. Acad. Sci. USA 93:3346-3351 (1996); Yao et al., Human Gene Therapy 10:419-427 (1999)). In general, these systems rely on the introduction of a gene for one or more exogenous switch proteins in addition to the therapeutic gene or the harnessing of one or more endogenous intracellular proteins. In the presence of the signal compound the protein(s) is activated to become either a positive or a negative regulator of transcription of the therapeutic gene. Because the protein must be constitutively expressed in order to be available for interaction with the signal compound, there is a threat of eliciting a cellular immune response, which could eliminate the cells carrying the therapeutic gene and killing essential tissue or cells. This in turn could lead to exacerbated or deleterious clinical situations.

One approach to reduce the immunogenicity of the protein has been to use naturally occurring proteins or fusions of these that bind to known signal compounds (See U.S. Pat. No. 6,187,757, entitled “Regulation of Biological Events Using Novel Compounds”). The method in this case utilizes the human FK506 binding protein as the signal binding protein. Unfortunately, the drugs known to bind this protein are highly active immuno-suppressive compounds (e.g. rapamycin) or incompletely characterized compounds with no prior clinical human experience.

Thus, there remains a need for a safe and efficient way to control transgene expression in patients undergoing gene therapy. The ability to control gene expression in a clinically acceptable fashion and in compliance with current FDA regulations would allow for the development of many effective gene therapies.

### SUMMARY OF THE INVENTION

The present invention provides an improved method for controlling gene expression in vivo through the use of a gene switch comprising one or more aptamers incorporated into the untranslated regions (UTR's) of the transgene of interest, including the 3′UTR , the 5′ UTR and between the genes of a multicistronic mRNA, and to compositions related to the same. The control of gene expression is provided by a ligand which binds to the aptamer, and the necessary condition to make this system function is to use either ligands that have sufficiently high binding efficiency to function with a single aptamer or to use more than one aptamer to arrive at the necessary affinities (<10-50 uM). The ligand is pre-chosen for certain (see detailed description) criteria and the aptamer is selected for that specific ligand. Regulation occurs by administering an effective dose of a ligand or modulator, preferably, a small molecule approved for human use by a national or international regulatory agency (in the USA this is the FDA). In a preferred embodiment, the small molecule or modulator shall have the capacity to cross the blood-brain barrier. Applications of such aptamers sequences incorporated into genes of interest in conjunction with specific preselected ligands include for “on” and “off” regulation of therapeutic transgenes, conditional replication of various gene delivery vectors, regulation of gene silencing, screening assays for drug antagonists or agonists, and othertherapeutic and diagnostic tests.

Thus, in a first embodiment, the present invention provides an expression vector comprising a transgene incorporating an aptamer into the 3′ untranslated region of the transgene or in an untranslated region between the transgenes of a multicistronic element. The aptamer can be of varying length and affinity for a selected modulator and is typically delivered to the patient through a viral or non-viral vector. The regulatable switch is created by the binding of the modulator to the aptamer to form a complex. The gene of interest may or may not be translated.

In another embodiment, the present invention provides a method for controlling transgene expression by incorporating one or more aptamers into an untranslated region of a transgene, and then providing a modulator capable of forming a complex with the aptamer to form a complex that controls the expression of the transgene. Regulation can be accomplished through the use of more than one aptamer, including aptamers of differing nucleotide sequences. The use of two, three or more different aptamers to the same modulator allows the use of individual aptamer sequences that individually lack the high affinity necessary to make this system usable in patients. This is important because it can often be difficult to find high affinity (<10 uM) ligand aptamer pairs (Werstuck and Green, op.cit), especially when the choice of ligands is limited to proven safe or approved compounds. Preferably, the modulator employed is a small molecule. Even more preferably, the small molecule is generally recognized as safe and is capable of crossing the blood brain barrier.

In a further embodiment the present invention provides a method for controlling transgene expression by using a three component system comprising an auxiliary protein, a modulator and an aptamer (FIG. 2). The auxiliary protein may be an endogenous protein that is naturally present at sufficient concentration within the target tissue, or an exogenous protein delivered by the same or a different vector to the one that delivers the transgene and aptamer. The modulator is selected to have high affinity for the auxililary protein. The aptamer component is chosen to have high affinity for the auxiliary protein-modulator complex but low affinity for the auxiliary protein alone. The advantage of this approach to the creation of a regulatory system is that the stability of the tripartite complex is likely to be higher than that of a single aptamer for modulator and therefore the likelihood of efficient regulation at concentrations of modulator that can be achieved in the tissue is increased. Regulation of expression is achieved by modifying the 5′UTR of the mRNA to contain the aptamer. Addition of ligand then drives formation of the tertiary complex on the RNA leading to downregulation of translation. In practical terms a control mechanism of this type relies on the existence of a ligand for a selected auxiliary protein, that is, an endogenous protein or exogenous protein. Preferably, when an exogenous protein is employed, the DNA encoding the same is delivered in the same vector as the transgene and aptamer of the present invention.

In yet another preferred embodiment, the present invention provides a method for treating a neurodegenerative disease in a mammal, especially a human patient, by administering an expression vector of the present invention to a target cell in the nervous system of the mammal. Expression of the transgene will be under the control of the aptamer, and regulated expression is achieved by administration of a small molecule capable of crossing the blood brain thereby providing a means of discontinuing treatment if that is clinically desirable.

In addition, the present invention also provides aptazymes which are ribozymes capable of cleaving sequences within their own or other transcript and which can be regulated as a result of binding ligand/modulator. The ribozyme may also be a self-cleaving ribozyme. As such they combine the properties of ribozymes and aptamers. Aptazymes offer advantages over conventional aptamers due to their potential for activity in trans, the fact that they act catalytically to inactivate expression and that inactivation, due to cleavage of their own or heterologous transcript, is irreversible.

The basic concept behind the use of an aptazyme is as follows. The aptazyme is included in an untranslated region of the genome and in the absence of ligand/modulator is inactive, allowing expression of the transgene. Expression can be turned off (or down-regulated) by addition of the ligand. It should be noted that aptazymes which are downregulated in response to the presence of a particular modulator could be used in control systems where upregulation of gene expression in response to modulator is required.

Aptazymes may also permit development of systems for self-regulation of transgene expression. For example, the transcript of a transgene whose protein product is the rate determining enzyme in synthesis of a particular small molecule could be modified to include an aptazyme selected to have increased catalytic activity in the presence of that molecule, thereby providing an autoregulatory feedback loop for its synthesis. Alternatively, the aptazyme activity could be selected to be sensitive to accumulation of a protein product, or any other cellular macromolecule.

These and various other advantages and features of novelty which characterize the invention are pointed out with particularity in the claims annexed hereto and forming a part hereof. However, for a better understanding of the invention, its advantages, and objects obtained by its use, reference should be made to the accompanying descriptive matter, in which there is illustrated and described preferred embodiments of the invention. These and other embodiments are disclosed or are obvious from and encompassed by, the following Detailed Description.

The terms “comprises”, “comprising”, and the like can have the meaning ascribed to them in U.S. Patent Law and can mean “includes”, “including” and the like.

### BRIEF DESCRIPTION OF THE DRAWINGS

FIG. 1 shows a generalized SELEX selection scheme. The process begins with a chemically synthesized random sequence DNA library. A library is designed to contain a contiguous randomized region (dashed lines) flanked by two fixed sequence regions. Each nucleotide position in the contiguous random region is synthesized upon delivery of a mixture of phosphoramidites containing all four building blocks: A, G, C, and T. Black arrows indicate steps in a DNA-based aptamer selection; gray arrows correspond to RNA-based selection. Tuerk et al., supra.

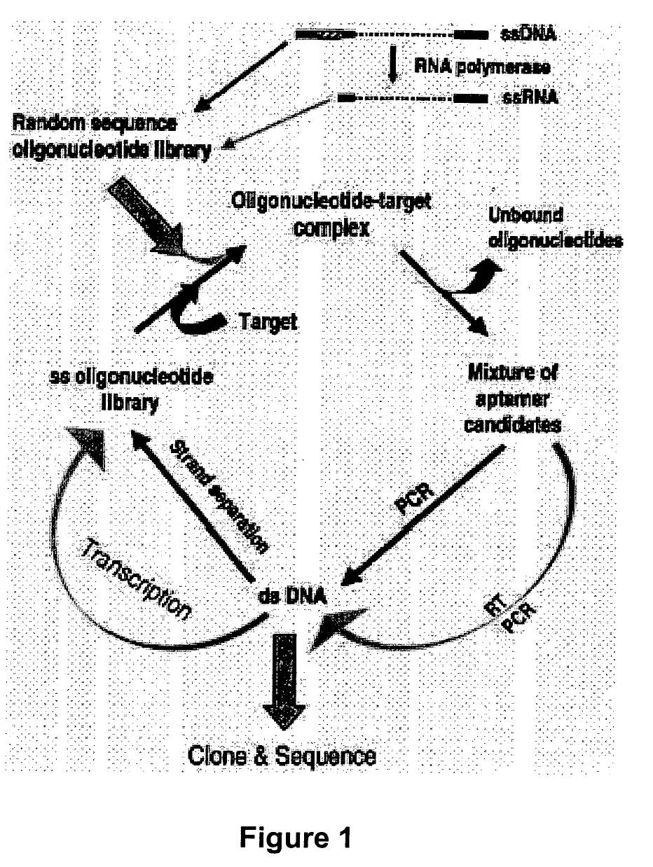

FIGS. 2A-B are schematics depicting the induction of different configurations and the regulation of expression by exploiting the interaction between a modulator-auxiliary protein complex and aptamer. FIG. 2A illustrates high affinity interaction between aptamer sequence/structure and auxiliary protein-modulator complex in the OFF state. FIG. 2B illustrates little to no interaction is between protein and aptamer sequence in the ON state.

FIGS. 3A-B are schematics depicting regulation of gene expression by an aptamer incorporated into the 3′ UTR of a gene of interest. FIG. 3A illustrates the ON state, and FIG. 3B illustrates the OFF state. In these constructs, a control mechanism is created by the ability of splicing to be regulated by placing an aptamer adjacent to the branchpoint of an intron. Splicing may then be inhibited by the presence of a stem-loop, formed by an aptamer in the presence of small molecule or modulator adjacent to the branch point. This switch mechanism can be incorporated into the transcript of the gene of interest from which expression is to be regulated by inclusion of a downstream synthetic intron which contains 1) a splice donor 2) an A/U-rich element (ARE) which causes transcript instability 3) an aptamer-branchpoint “switch” and 4) a splice acceptor.

FIGS. 4A-B are schematics depicting downregulation of a gene of interest by use of a gene switch comprising a small molecule or modulator bound to an aptamer and a stabilizing anti-aptamer. FIG. 4A illustrates the ON state, and FIG. 4B illustrates the OFF state.

FIGS. 5A-B are schematics depicting a gene switch comprising an IRES (internal ribosome entry site) flanked by a small molecule or modulator bound to aptamer A and a stabilizing anti-aptamer B. FIG. 5A illustrates the OFF state, and FIG. 5B illustrates the ON state.

FIGS. 6A-B are schematics depicting downregulation of gene expression by an aptazyme in the 3′ UTR. FIG. 6A illustrates expression of the gene of interest, whereas FIG. 6B illustrates a reduction of gene. Binding of the small molecule or modulator triggers a conformational change activating the self-cleaving ribozyme activity, and removal of the poly(A) tail resulting in RNA degradation and a reduction in gene expression.

FIG. 7 illustrates modulation of a therapeutic gene of interest (i.e., insulin) in which the transcript is cleaved above threshold glucose level and no translation of insulin occurs.

### DETAILED DESCRIPTION OF THE INVENTION

The synthesis of RNA using DNA as a template is called transcription. Transcription involves the synthesis of an RNA molecule that is complementary in base sequence to the coding strand of the DNA duplex. Chain elongation occurs as the RNA polymerase moves along the DNA molecule (coding strand) from the 3′ to the 5′ end. Because nucleotide pairing is antiparallel, the RNA strand (mRNA) is elongated in the 5′ to 3′ direction as each successive nucleotide is added to the 3′ end of the growing chain.

By contrast, the process of protein synthesis is called translation. The main elements of the translation apparatus are the mRNA to be translated, the ribosomes responsible for the process, the tRNA responsible for carrying activated amino acids to their correct locations, the aminoacyl-tRNA synthetases that add amino acids to their appropriate tRNA molecules, and many protein factors that participate at several stages of the translation process.

In their most basic form, the regulatable gene cassettes of the present invention comprise a transgene incorporating an aptamer based control mechanism located in the 3′ untranslated region of the transgene or in an untranslated region between the transgenes of a multicistronic cassette. A specific example of control by the use of A/U-rich regions and a self-cleaving intron is discussed below. Regulation or control of the expression of the transgene(s) is accomplished through the binding of a ligand or modulator compound to the aptamer. The interaction between the aptamer and the modulator is reversible, thus collectively these components provide a “gene switch” for controlling the expression of the transgene.

Aptamers

As used herein, “aptamers” are oligonucleotide sequences with the capacity to recognize virtually any class of target molecules with high affinity and specificity. In a preferred embodiment, the aptamer specifically binds to a modulator capable of crossing the blood-brain barrier that is or will be approved for human use. As used herein, “Oligonucleotide” refers to a molecule comprised of the deoxyribonucleotides derived from the bases adenine (A), guanine (G), thymine (T) and/or cytosine (C) or analogues of these, in either single-stranded form or a double-stranded helix, and comprises or includes an “aptamer” according to the present invention, as that term is defined herein. Thus, the term “oligonucleotide” includes double-stranded DNA found in linear DNA molecules or fragments of DNA, viruses, plasmids, vectors, chromosomes or synthetically derived DNA. The term oligonucleotide also includes molecules comprised of ribonucleotides such as those derived from the bases adenine, guanine, uracil and/or cytosine, or analogues of these. Libraries of RNA aptamers can be created enzymatically from chemically synthesized DNA aptamer libraries by using in vitro transcription techniques.

The optimal length of the oligonucleotide sequence in the aptamer will vary, depending on factors including, but not limited to, the size, shape, charge, and hydrophilic properties of the modulator. Typically, the aptamer will be from about 10 to about 200 base pairs in length, preferably from about 20 to about 100 nucleotides in length, more preferably from about 20 to about 60 nucleotides in length, and most preferably from about 20 to about 40 nucleotides in length. The molecular context of an aptamer can affect the activity of the aptamer (R. E. Martell et al. Mol Ther. 6:30-34 (2002) and so, for example, not all oligonucleotide sequences around the aptamer will be suitable for use in the current invention.

Selection of the Aptamer

Techniques for the selection of aptamers that bind specifically to a modulator useful in the compositions and methods of the present invention, are known in the art. Those techniques can be employed routinely to obtain an essentially unlimited number of aptamers useful in the present invention. Examples of publications containing useful information on in vitro selection of aptamers include the following: Flug et al., Molecular Biology Reports 20:97-107 (1994); Wallis et al., Chem. Biol. 2:543-552 (1995); Ellington, Curr. Biol. 4:427-429; Lato et al., Chem. Biol. 2:291-303 (1995); Conrad et al., Mol. Div. 1:69-78 (1995); and Uphoff et al., Curr. Opin. Struct. Biol. 6:281-284 (1996).

The basic steps in conventional in vitro selection of an aptamer are as follows. A random DNA pool is synthesized, i.e., a pool of DNA molecules having random nucleotide sequences. The random DNA pool is transcribed in vitro to produce a random RNA pool. The RNA pool is subjected to affinity chromatography using a column matrix to which the modulator is immobilised. RNA molecules that bind specifically to the immobilized modulator are collected and reverse-transcribed into cDNA and amplified by PCR. The PCR-amplified products are then transcribed into RNA. This process is repeated for as many cycles as necessary to yield a population of nucleic acid molecules that bind to the modulator with the desired affinity (and specificity).

Individual nucleic acid molecules from the selected population are cloned and sequenced using conventional recombinant DNA technology. Such technology is described in numerous references, e.g., Sambrook et al., Molecular Cloning—A Laboratory Manual (2nd ed.), Cold Spring Harbor Laboratory Press (1989).

A preferred method for selecting aptamers useful in the composition and methods of the present invention is commonly referred to as the “SELEX” (Systematic Evolution of Ligands by Exponential Enrichment ) method. Tuerk et al., Science 219:505-510 (1990), S. D. Jayasena Clinical Chemistry 45:1628-1650 (1999). SELEX allows for the screening of large random pools of nucleic acid molecules for a particular functionality. This technique has been used to screen for various functionalities, such as binding to small organic molecules. (Famulok et al., Am. J. Chem. Soc. 116:1698-1706 (1994); Connell et al., Biochemistry 32:5497-5502 (1194); Ellington et al., Nature 346:818-822 (1990); binding to large proteins (Jellinek et al., Proc. Natl. Acad. Sci. USA 90:11227-11231 (1993); Tuerk et al., Proc. Natl. Acad. Sci. USA 89:6988-6992 (1992); Tuerk et al., Gene 137:33-39 (1993); Schneider et al., J. Mol. Biol. 228:862-869 (1992)) and the alteration or de novo generation of ribozymes (Liu et al., Cell 77:1093-1100 (1994); Green et al., Nature 347:406-408 (1990); Green et al., Science 258:1910-1915 (1992); Pun et al., Biochemistry 31:3887-3895 (1992); Bartek et al., Science 261:1411-1418 (1993). The aptamers are selected by column chromatography or any other technique of enrichment for the desired function.

Incorporation of the Aptamer

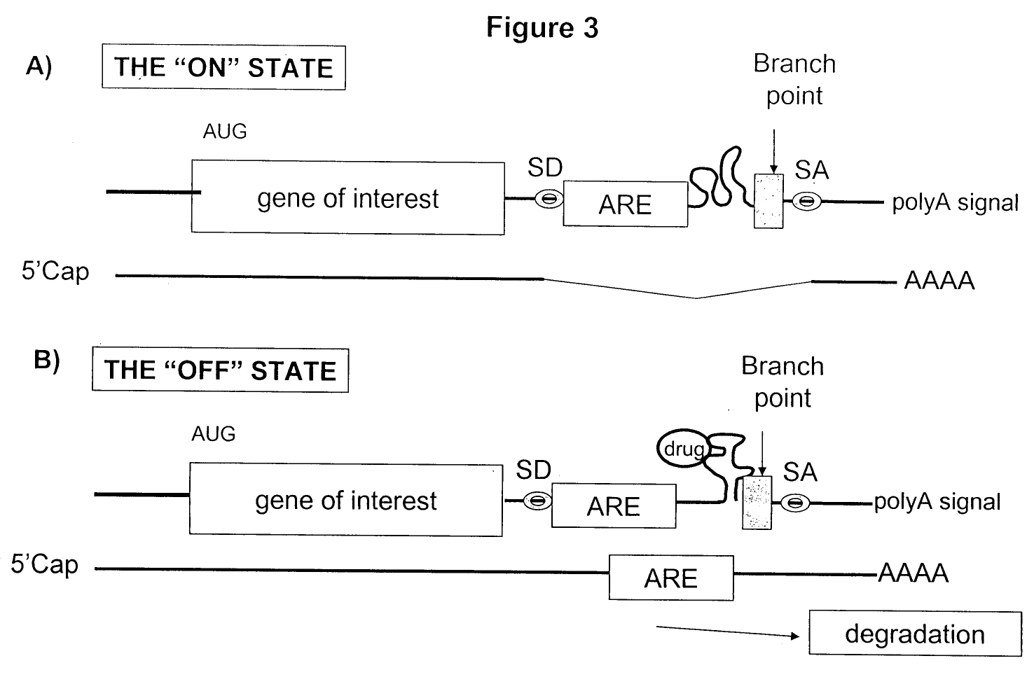

An aptamer(s) may be operably linked to a nucleotide sequence of interest. The term “operably linked” means that the components described are in a relationship permitting them to function in their intended manner. In a preferred embodiment, an aptamer(s) may be incorporated in the 5′ or 3′ region of a nucleotide sequence of interest or in the 5′ or 3′ UTR of a gene of interest. In a preferred embodiment, the aptamer is incorporated in the 3′ UTR of the gene of interest (FIGS. 3A-B). For example, a control mechanism can be constructed by utilizing the ability of splicing to be regulated by placing an aptamer adjacent to the branchpoint of an intron. In this scenario, splicing can be inhibited by the presence of a stem-loop, formed by an aptamer in the presence of ligand/modulator adjacent to the branch point. This switch mechanism can be incorporated into the transcript of the gene from which expression is to be regulated by inclusion of a downstream synthetic intron which contains 1) a splice donor 2) an A/U-rich element (ARE)(which causes transcript instability 3) an aptamer-branchpoint “switch” and 4) a splice acceptor. In the absence of ligand the splicing machinery is operative and the transcript is stable (due to the exclusion of the ARE). However, in the presence of ligand, splicing is inhibited hence the transcript contains the ARE and is relatively destabilized leading to a reduced level of expression. The regulated intron can also be placed in the 5′UTR.

Aptamers may also be incorporated into the untranslated region between the transgenes of a multicistronic cassette, or in both the 5′ and 3′ UTRs, thereby increasing the sensitivity, and therefore, regulatability of the gene switch. The construction of a multicistronic or otherwise known as a polycistronic cassette involves the use of one or more internal ribosome entry sites (IRES's), Construction of polycistronic vectors are known in the art and can be found, for example, in U.S. Pat. No. 6,319,707, incorporated herein by reference. An IRES may be derived from a virus, e.g., a picornavirus such as encephalomyocarditis virus (EMCV) or polivirus (PV) (Ghattas, I. R., et al., Mol. Cell. Biol., 11:5848-5859, 1991; Pelletier and Sonenberg, Nature 334: 320-325, 1988), or from a cellular gene, e.g., FGF-2 and NRF (Creancieret al., J Cell Biol, 150: 275-281, 2000; Oumard et al., Mol Cell Biol, 20: 2755-2759).

In any construct utilizing an internal cassette having more than one IRES and gene, the IRESs may be of different origins, that is, heterologous to one another. For example, one IRES may be from EMCV and the other IRES may be from PV.

Although IRESs are an efficient way to co-express multiple genes from one vector, other methods are also useful, and may be used alone or in conjunction with IRESs. These include the use of multiple internal promoters in the vector (Overell et al., Mol Cell Biol. 8: 1803-8 (1988)), the use of alternate splicing patterns leading to multiple RNA species derived from the single viral genome that expresses the different genes (Cepko et al. Cell 37: 1053 (1984)) or simply appropriately spacing the multicistronic genes (Levine F, Yee J K, Friedmann T, Gene.; 108:167-74 (1991)).

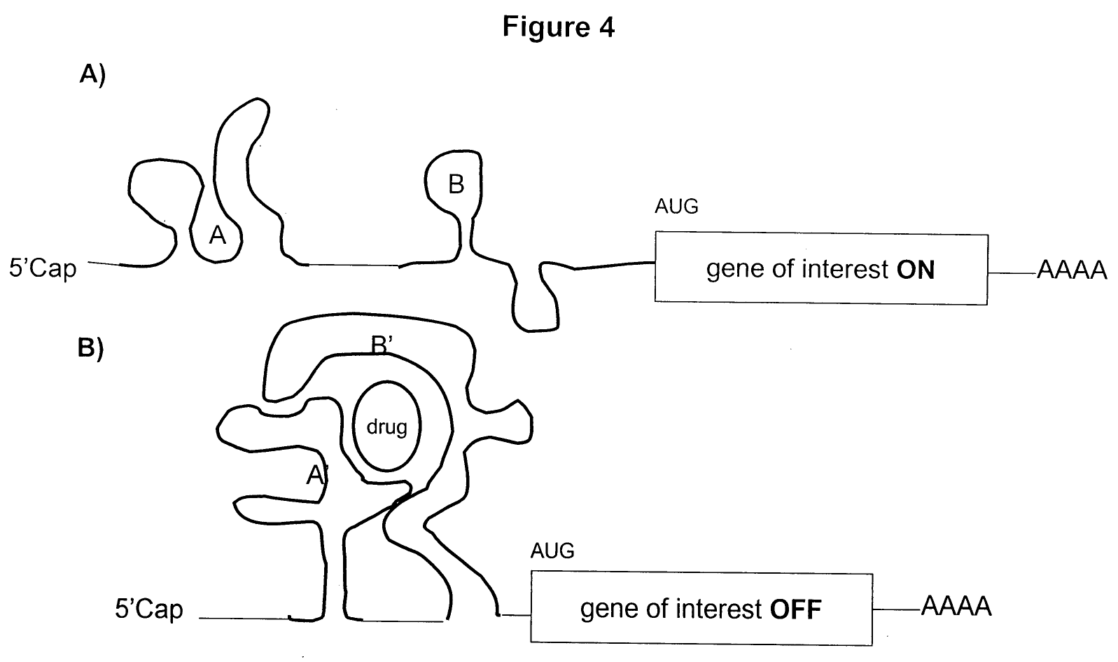

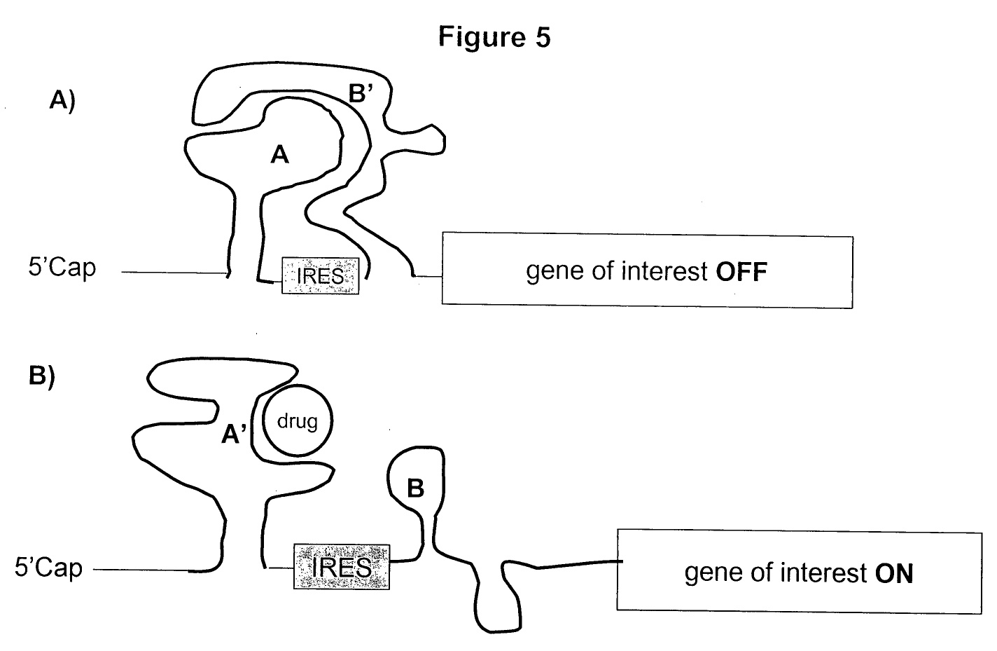

In another embodiment, two or more aptamers of distinct nucleotide sequences are incorporated into the 3′ UTR of the transgene or into the UTR between the transgenes of a multicistronic cassette. Typically, the two or more aptamers will be located adjacent to each other and joined by an appropriate linker element. The inclusion of a linker element is important in maintaining the functional integrity of the aptamers. In this embodiment, the distinct aptamers may have varying affinities for the modulator, or for different modulators. For example, the first aptamer (A) and the modulator may form a complex with relatively high affinity, however a second aptamer (B) when alone may only have only a low affinity for ligand or modulator. However, aptamer B may have a high affinity for the aptamer A—ligand/modulator complex. Accordingly, the resultant complex provides much greater affinity and stability than either of the aptamers complexed with the modulator individually, thereby improving the regulation that can be achieved (FIGS. 4A-B). In another embodiment an “on-off” system of regulation of gene expression is achieved. In the off state there is a strong interaction between two aptamer sequences, the 5′ of which is a high affinity aptamer which flank an IRES and this interaction is sufficient to inhibit translation directed by the IRES. The “on” state is activated by addition of ligand or modulator which disrupts the interaction of the two aptamer sequences by binding to the aptamer (FIGS. 5A-B).

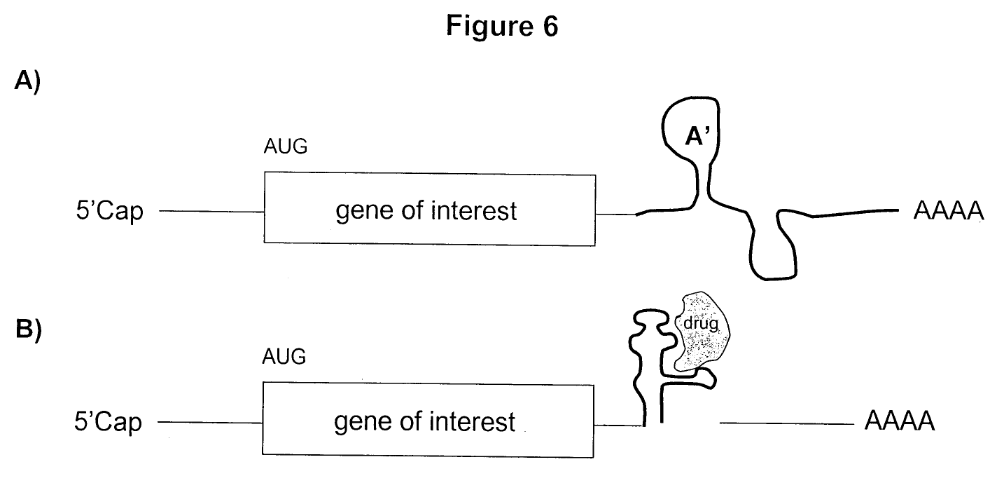

Multimers of a single aptamer or the combination of two or more aptamers of distinct nucleotide sequences can also be incorporated into the 5′ UTR of a transgene according to the present invention. In another embodiment of the present invention, the aptamer sequence may be an aptazyme (FIGS. 6A-B), s obtained either de novo as a result of screening a library of aptamers for appropriate RNA cleavage activity in the presence of modulator or produced by replacing a helix of a ribozyme with an aptamer. In general the structure of ribozymes is fairly plastic and it is relatively straightforward to construct functional ribozymes with a desired sequence incorporated therein (Hermann,T. and Patel D. J., J. Mol. Biol. (1999) 294, 829-849).

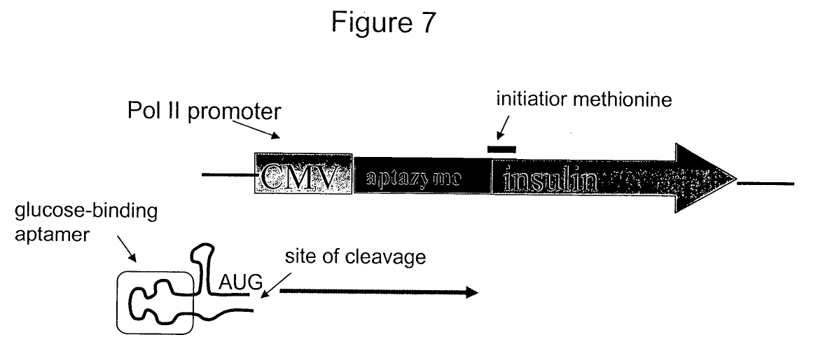

The use of aptazymes encompasses post-transcriptional regulation of a nucleic acid sequence, including for example, therapeutic gene sequences, antisense sequences, or short hairpin RNA sequences. The aptazyme may be activated (or inhibited) by the addition/removal of the appropriate modulator or small molecule which induces cleavage of the transcript and thus, blocks synthesis of a gene product. This may be accomplished by the aptazyme functioning to remove a part of the transcript, for example, the codon initiator methionine, or the 5′ cap or polyadenylate tract of the transcript. These processes would shut off synthesis of a gene product if expression levels become too high. It can be seen that the level of expression of the transgene would be self-regulating if the modulator/ligand was a product whose synthesis was brought about either directly or indirectly by the product of the transgene. For example, an aptazyme which was modulated by dopamine would be useful to control synthesis of a transgene encoding aromatic acid amino acid decarboxylase (MDC), the enzyme which synthesizes dopamine from L-dopa. A second example in which glucose is used as a modulator is as follows. An aptazyme whose activity is modulated by glucose binding could be designed such that high level expression of insulin occurs only when blood glucose levels are high. If glucose falls below a threshold level then the aptazyme would be active and the insulin transgene transcript would not be permitted to complete synthesis of the insulin gene product (FIG. 7) (GB 0130797.4; GB 0201140.1; GB 0211409.8; U.S. Ser. No. 10/082,122).

The examples above relate to the situation in which the activity of the aptazyme is activated by addition of ligand/modulator. However, for some applications it might be desirable to have the gene of interest expressed at a low level and inducibly upregulated by a modulator. This is achieved by changing the selection process of the aptazyme which is described in example 3 herein. In this modification the first round of selection will be for aptazymes which are not cleaved in the presence of the inhibitor. The second step is a positive selection for self-cleaving aptazymes, after the ligand is washed out. This may involve denaturing and renaturing of the aptazyme if the ligand is bound with a high affinity that won't allow dissociation of the small molecule or modulator from the aptazyme.

In accordance with the invention, standard molecular biology techniques may be used which are within the level of skill in the art. Such techniques are fully described in the literature. See for example; Sambrook et al. (1989) Molecular Cloning; a laboratory manual; Hames and Glover (1985-1997) DNA Cloning: a practical approach, Volumes I-IV (second edition); Methods for the engineering of immunoglobulin genes are given in McCafferty et al. (1996) “Antibody Engineering: A Practical Approach”.

Modulator

Virtually any compound can be utilized as a modulator in the present invention. The use of the term “modulator” may refer to any of a ligand, small molecule or drug which possesses the ability to bind to an aptamer(s) of the present invention. As used herein, “modulator” refers to a wide range of compounds or compositions, including, but not limited to natural, synthetic or semi-synthetic organic molecules, proteins, oligonucleotides and antisense, that form a complex with the aptamer(s) of the present invention. Furthermore, the precursor of a modulator (i.e., a compound that can be converted into a modulator) is also considered to be a modulator. Similarly, a compound which converts a precursor into a modulator is also considered to be a modulator. One or more of the following criteria for choosing a modulator are preferred:
1) Orally (or other nonparenteral route) available to allow blood concentrations of 10 ng/ml or higher.
2) Approved drug (OTC (over-the-counter) or prescription) or at least manufactured in a standardized way.
3) Clinically acceptable safety profile (taken by many people).
4) Large enough to allow aptamer discrimination (MW>250)
5) Able to cross blood brain barrier or other known anatomical compartment barriers as needed to reach the delivered genes
6) Able to cross cell membrane
7) Pharmacologically inert (or with well-known mild action)
8) No or limited toxicity
9) Usable half life (>6 hours preferably) in serum and in the cell.

The modulator must bind an aptamer with suitable affinity and specificity. Whether a molecule will bind to an aptamer with suitable affinity and specificity depends on factors including molecular size, shape, charge and hydrophilic properties of the modulator. In this regard, the dissociation constant (K_{d}) of the modulator for the aptamer should be less than about 10 uM, preferably less than about 1 uM, more preferably less than about 100 nM, even more preferably less than 10 nM, even more preferably less than 1 nM and most preferably less than 0.1 nM.

Preferably, the modulator can be readily formulated into a therapeutic agent. Examples of the classes of compounds from which modulators useful in the practice of the present invention may be chosen include, but are not limited to, steroids, sterols, retinoids, prostaglandins, leukotrienes, thiazolidinediones, farnesoids, aminobenzoates, hydroxybenzoates, eicosanoids, cholesterol metabolites, fibrates, amino acids, sugars, nucleotides, fatty acids, lipids, serotonin, dopamine, catecholamines, acid azoles, and the like. Specific compounds or formulations that can be used include: those on the “Generally recognized as safe (GRAS) list (available from the FDA); OTC medicines including analgesics such as aspirin, ibuprofen or paracetamol or their derivatives in normal or slow release formulations such as Ecotrin, manufactured by Glaxo Smith Kline; (see the Physicians Desk Reference, 2002); artificial sweetners such as saccharin (Renwick A. G., Food Chem Toxicol. 23:429-435 (1985)) or aspartame (Romano,D. et al. Food Chem Toxicol 28:317-321 (1990)); commonly used anticoagulants such as low molecular weight heparin (Duplaga et al. Pharmacotherapy 21:218-234 (2001); orally available anti-herpetics such as valacyclovir (GlaxoSmithKline) or Valcyte (Roche) (see Physician's Desk Reference op.cit) D-amino acids such as D-Arginine, nicotine in various formulations (Hajek P, et al. Arch Intern Med.; 159:2033-8, 1999) including nicotine patches where the nicotine is absorbed through the skin; Nitroglycerin; well characterized food additives such as vitamins including vitamin C, topical creams such as Retin A; anti-ulcer drugs such as Tagamet (H2 receptor antagonists); nucleoside analogues including antifungal drugs such as fluorocytidine; antibiotics such as tetracylin, doxycyclin, streptomycin, erythromycin. Using an approved drug can be advantageous because information on safety, side effects, dosage, route of administration, pharmacokinetics, metabolism, clearance and other useful information is available. In addition it is illegal to sell, for clinical use, any kind of drug without regulatory approval. Preferred drugs are those that display mild pharmacological activities and minimal side effects.

Preferably, the modulator displays low toxicity so that unwanted biological side effects are minimized. More preferably, the modulator also is capable of crossing the blood-brain barrier. When the cell containing the gene is in vivo, the modulator is chosen to have an in vivo persistence sufficient to allow an effective amount of the modulator to reach and enter the cell.

By following the above rules and guidance, it is possible to choose agents that are already acceptable for human use and that will have tolerable side-effects while being used as a modulatory agent for controlling gene expression from an expression vector.

It is not necessary for the modulator to be a drug. In preferred embodiments of the invention, the modulator is pharmacologically inert (except for s its activity in binding the aptamer according to this invention). Preferably, the modulator is an organic compound. The design and synthesis of small, organic, cell-permeable molecules useful as modulators in this invention are described, for example, in Amara et al., Proc. Antl. Acad. Sci. USA 94:10618-10623 (1007); and Keenan et al., Bioorganic & Medicinal Chemistry 6:1309-1335 (1998). Modulators according to the present invention can be prepared as pharmaceutically acceptable salts. Examples of pharmaceutically acceptable salts include acid addition salts such as those containing hydrochloride, sulfate, phosphate, sulfamate, acetate, citrate, lactate, tartrate, methanesulfonate, ethanesulfonate, benzenesulfonate, p-toluenesulfonate, cyclohexylsulfamate and quinate. (See e.g., PCT/US92/03736). Such salts can be derived using acids such as hydrochloric acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, and quinic acid.

Pharmaceutically acceptable salts can be prepared by standard techniques. For example, the free base form of the compound is first dissolved in a suitable solvent such as an aqueous or aqueous-alcohol solution, containing the appropriate acid. The salt is then isolated by evaporating the solution. In another example, the salt is prepared by reacting the free base and acid in an organic solvent.

Carriers or excipients can be used to facilitate administration of the modulator, for example, to increase the solubility of the compound. Examples of carriers and excipients include calcium carbonate, calcium phosphate, various sugars or types of starch, cellulose derivatives, gelatin, vegetable oils, polyethylene glycols and physiologically compatible solvents.

The modulator can be used as formulated by the manufacturer or formulated, individually or in combination, into pharmaceutical compositions by admixture with pharmaceutically acceptable non-toxic exipients and carriers. Such compositions can be prepared for use in parenteral administration, in the form of liquid solutions or suspensions; for oral administration in the form of liquid, tablets or capsules; or intranasally in the form of powders, nasal drops or aerosols.

Gene Switch

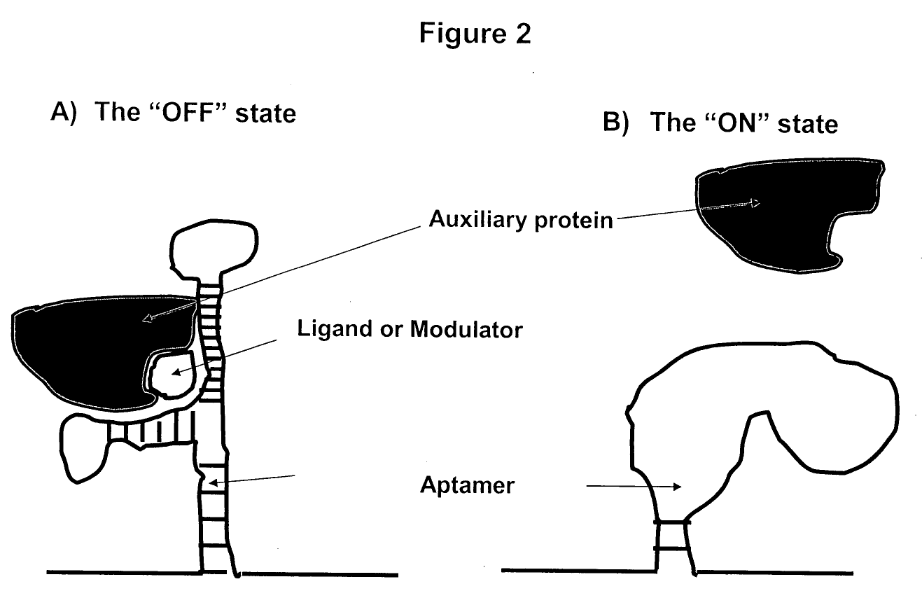

In a preferred alternative embodiment, the gene switch complex further comprises an auxiliary protein (FIG. 2). The auxiliary protein modifies, and preferably enhances the binding of the modulator to the aptamer in vivo. Accordingly, the auxiliary protein can be an endogenous protein present in sufficient concentration within the target cell(s). Alternatively, the auxiliary protein can be exogenous to the target cell, and will typically be delivered via an appropriate vector into the target cell(s) and expressed therein. Preferably, the DNA encoding the exogenous protein is delivered in the same vector utilized to deliver the gene switch cassette of the present invention. Preferably, the exogenous protein is one that is a “self′ protein for the human or animal being treated, to avoid immunological issues.

As with the use of multiple aptamers, this embodiment provides for a more refined and specific degree of control over the expression of the transgene of interest. For example, this embodiment would allow for the use of smaller aptamers which, by themselves, would typically not provide the binding affinity desireable in the methods and compositions of the present invention. However, when the aptamer is complexed with the auxiliary protein, the combination provides a highly specific and regulatable substrate that complexes with the selected modulator. Further, careful selection of the auxiliary protein, whether endogenous or exogenous, will provide the needed specificity to the gene switch complex, since such complex will only form and function in the cells and tissues where the auxiliary protein is expressed.

When utilized to control gene expression in vitro, the gene switch cassettes of the present invention can be placed in virtually any cell background by techniques well known to those of ordinary skill in the art, for example, transfection, infection, or electroporation. As used herein, the term “cell” refers to a prokaryotic cell or a eukaryotic cell. In the more preferred embodiment, the eukaryotic cell is a mammalian cell.

Vector

An expression vector is used which is capable of delivering a nucleotide sequence of interest to a cell. Such an expression vector is capable of transfecting, transducing or infecting a cell, dividing or non-dividing. Preferably, the transgene under the regulatory control of the aptamer is delivered by a vector capable of infecting non-dividing and/or slowly-dividing cells. Mammalian non-dividing and slowly-dividing cells include brain cells, stem cells, terminally differentiated cells, such as macrophages, lung epithelial cells and various other cell types. Dividing cells may be tumor cells, for example, and may further include tumor cells which are slowly dividing cells. In particular, target cells for gene therapy using retroviral vectors include but are not limited to haematopoietic cells, (including monocytes, macrophages, lymphocytes, granulocytes, or progenitor cells of any of these); endothelial cells, tumor cells, stromal cells, astrocytes, or glial cells, muscle cells, epithelial cells, neurons, fibroblasts, hepatocytes, astrocytes, kidney cells, liver cells, heart and lung cells.

As it is well known in the art, a vector is a tool that allows or faciliates the transfer of an entity from one environment to another (See for example “The Development of Human Gene Therapy” T. Friedmann Ed., 1999 Cold Spring Harbor Press). By way of example, some vectors used in recombinant DNA techniques allow entities, such as a segment of DNA (such as a heterologous transgene) to be transferred into a target cell. Optionally, once within the target cell, the vector may then serve to maintain the transgene within the cell or may act as a unit of DNA replication. Examples of vectors used in recombinant DNA techniques include plasmids, chromosomes, artificial chromosomes or viruses. The vector of the present invention may be delivered to a target site by a non-viral or a viral vector.

Non-viral delivery systems include but are not limited to DNA transfection methods. Here, transfection includes a process using a non-viral vector to deliver a gene to a target eukaryotic cell such as a mammalian cell.

Typical transfection methods include direct DNA injection, electroporation, DNA biolistics, lipid-mediated transfection, compacted DNA-mediated transfection, liposomes, immunoliposomes, lipofectin, cationic agent-mediated, cationic facial amphiphiles (CFAs) (Nature Biotechnology 1996 14; 556), and combinations thereof.

Viral delivery systems include but are not limited to adenovirus vector, adeno-associated viral (AAV) vector, a herpes viral vector, retroviral vector, lentiviral vector, and baculoviral vector. Other examples of vectors include ex vivo delivery systems, which include but are not limited to DNA transfection methods such as electroporation, DNA biolistics, lipid-mediated transfection, compacted DNA-mediated transfection.

In a preferred embodiment, the vector comprises a retroviral vector. The term “retroviral vector particle” refers to the packaged retroviral vector, that is preferably capable of binding to and entering target cells. There are many retroviruses. For the present application, the term “retrovirus” includes: murine leukemia virus (MLV), human immunodeficiency virus (HIV), equine infectious anaemia virus (EIAV), mouse mammary tumour virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV) and all other retroviridiae including lentiviruses. A detailed list of retroviruses may be found in Coffin et al. (“Retroviruses” 1997 Cold Spring Harbour Laboratory Press Eds: J M Coffin, S M Hughes, H E Varmus pp 758-763).

Retroviruses may be broadly divided into two categories: namely, “simple” and “complex”. Retroviruses may even be further divided into seven groups. Five of these groups represent retroviruses with oncogenic potential. The remaining two groups are the lentiviruses and the spumaviruses. A review of these retroviruses is presented in Coffin et al., 1997 (ibid).

A distinction between the lentivirus family and other types of retroviruses is that lentiviruses have the capability to infect both dividing and non-dividing cells (Lewis et al. 1992 EMBO. J 11: 3053-3058; Lewis and Emerman 1994 J. Virol. 68: 510-516). In contrast, other retroviruses—such as MLV—are unable to infect non-dividing cells such as those that make up, for example, muscle, brain, lung and liver tissue.

A lentiviral or lentivirus vector, as used herein, is a vector which comprises at least one component part derivable from a lentivirus. Preferably, that component part is involved in the biological mechanisms by which the vector infects cells, expresses genes or is replicated. The term “derivable” is used in its normal sense as meaning the sequence need not necessarily be obtained from a retrovirus but instead could be derived therefrom. By way of example, the sequence may be prepared synthetically or by use of recombinant DNA techniques.

More preferably, the retroviral vector is a lentiviral vector, as the lentivirus is known to infect non-dividing cells as well as slowly dividing cells. Examples of lentiviral vectors derived from HIV, EIAV, and FIV are described in many publications, for example, Naldini et al., (1996), Science. 272, 263-7; Mitrophanous et a/., (1999), Gene Ther. 6, 1808ff; Olsen, J. (1998), Gene Ther. 5, 1481ff, Buchschacher G L Jr, Wong-Staal F. Blood. 95:2499-504, 2000).

The components of the retroviral vector particle may be modified with respect to the wild type retrovirus. For example, the Env proteins in the proteinaceous coat of the particle may be genetically modified or replaced in order to alter their targeting specificity or achieve some other desired function.

Preferably, the viral vector preferentially transduces a certain cell type or cell types. More preferably, the viral vector is a targeted vector, that is, it has a tissue or cellular tropism which is altered compared to the native virus, so that the vector is targeted to particular cells.

Preferably the envelope is one which allows transduction of human cells. Examples of suitable env genes include, but are not limited to, VSV-G, an MLV amphotropic env such as the 4070A env, the RD114 feline leukaemia virus env or haemagglutinin (HA) from an influenza virus. The Env protein may be one which is capable of binding to a receptor on a limited number of human cell types and may be an engineered envelope containing targeting moieties. The env and gag-pol coding sequences are transcribed from a promoter and optionally an enhancer active in the chosen packaging cell line and the transcription unit is terminated by a polyadenylation signal. For example, if the packaging cell is a human cell, a suitable promoter-enhancer combination is that from the human cytomegalovirus major immediate early (hCMV-MIE) gene and a polyadenylation signal from SV40 virus may be used. Other suitable promoters and polyadenylation signals are known in the art.

The packaging cell may be an in vivo packaging cell in the body of an individual to be treated or it may be a cell cultured in vitro such as a tissue culture cell line. Suitable cell lines include mammalian cells such as murine fibroblast derived cell lines or human cell lines. Preferably the packaging cell line is a human cell line, such as for example: 293 cell line, HEK293, 293T, TE671, HT1080.

Alternatively, the packaging cell may be a cell derived from the individual to be treated such as a monocyte, macrophage, stem cell, blood cell or fibroblast. The cell may be isolated from an individual and the packaging and vector components administered ex vivo followed by re-administration of the autologous packaging cells. Alternatively the packaging and vector components may be administered to the packaging cell in vivo. Methods for introducing retroviral packaging and vector components into cells of an individual are known in the art. For example, one approach is to introduce the different DNA sequences that are required to produce a retroviral vector particle e.g., the Env coding sequence, the gag-pol coding sequence and the defective retroviral genome into the cell simultaneously by transient triple transfection (Landau & Littman 1992 J. Virol. 66, 5110; Soneoka et al. 1995 Nucleic Acids Res 23:628-633).

In a preferred embodiment for transduction of slowly dividing or non-dividing cells, the retroviral vector is a lentiviral vector. The lentiviral vector is advantageous, as described in U.S. Pat. Nos. 6,312,683; 6,312,682; and 6,277,633, all of which are incorporated herein by reference. The lentivirus group can be split into “primate” and “non-primate”. Examples of primate lentiviruses include the human immunodeficiency virus (HIV), the causative agent of human acquired immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV) and the more recently described feline immunodeficiency virus (FIV) and bovine immunodeficiency virus (BIV).

Details on the genomic structure of some lentiviruses may be found in the art. By way of example, details on HIV and EIAV may be found from the NCBI Genbank database (i.e. Genome Accession Nos. AF033819 and AF033820 respectively).

Each retroviral genome comprises genes called gag, pol and env which code for virion proteins and enzymes. These genes are flanked at both ends by regions called long terminal repeats (LTRs). The LTRs are responsible for proviral integration, and transcription. They also serve as enhancer-promoter sequences. In other words, the LTRs can control the expression of the viral genes. Encapsidation of the retroviral RNAs occurs by virtue of a psi sequence located at the 5′ end of the viral genome.

The LTRs themselves are identical sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

For the viral genome, the site of transcription initiation is at the boundary between U3 and R in the left hand side LTR and the site of poly (A) addition (termination) is at the boundary between R and U5 in the right hand side LTR. U3 contains most of the transcriptional control elements of the provirus, which include the promoter and multiple enhancer sequences responsive to cellular and in some cases, viral transcriptional activator proteins. Some retroviruses have any one or more of the following genes that code for proteins that are involved in the regulation of gene expression: tat, rev, tax and rex.

With regard to the structural genes gag, pol and env themselves, gag encodes the internal structural protein of the virus. Gag protein is proteolytically processed into the mature proteins MA (matrix), CA (capsid) and NC (nucleocapsid). The pol gene encodes the reverse transcriptase (RT), which contains DNA polymerase, associated RNase H and integrase (IN), which mediate replication of the genome. The env gene encodes the surface (SU) glycoprotein and the transmembrane (TM) protein of the virion, which form a complex that interacts specifically with cellular receptor proteins. This interaction leads ultimately to infection by fusion of the viral membrane with the cell membrane.

The construction of the retroviral vector may also involve pseudotyping. The term pseudotyping means incorporating in at least a part of, or substituting a part of, or replacing all of, an env gene of a viral genome with a heterologous env gene, for example an env gene from another virus. Pseudotyping is not a new phenomenon and examples may be found in WO 99/61639, WO-A-98/05759, WO-A-98/05754, WO-A-97/17457, WO-A-96/09400, WO-A-91/00047 and Mebatsion et al. 1997 Cell 90, 841-847.

In a preferred embodiment of the present invention the vector system is pseudotyped with a gene encoding at least part of the rabies G protein. In a further preferred embodiment of the present invention the vector system is pseudotyped with a gene encoding at least part of the VSV-G protein (Burns et al. PNAS, 90: 8033-3087, 1993). In a further preferred embodiment, the vector system is pseudotyped with a gene encoding at least part of an RD114 protein (Cosset et al. J of Virol, 69: 7430-7436.) In a further preferred embodiment, the vector system is pseudotyped with a gene encoding at least part of a Chandipura G protein (Masters et al., Virology, 171: 285-290, 1989).

Retroviruses may also contain “additional” genes which code for proteins other than gag, poland env. Examples of additional genes include in HIV, one or more of vif, vpr, vpx, vpu, tat, rev and nef. EIAV has, for example, the additional genes S2, rev and tat. In addition, and in common with the other non-primate lentiviruses, it also encodes a dUTPase function within the pol polyprotein.

Proteins encoded by additional genes serve various functions, some of which may be duplicative of a function provided by a cellular protein. In EIAV, for example, tat acts as a transcriptional activator of the viral LTR. It binds to a stable, stem-loop RNA secondary structure referred to as TAR. Rev regulates and coordinates the expression of viral genes through rev-response elements (RRE). The mechanisms of action of these two proteins are thought to be broadly similar to the analogous mechanisms in the primate lentiviruses. The function of S2 is unknown. In addition, an EIAV protein, Ttm, has been identified that is encoded by the first exon of tat spliced to the env coding sequence at the start of the transmembrane protein.

It has been demonstrated that a lentivirus minimal system can be constructed from HIV, SIV, FIV, and EIAV viruses. Such a system requires none of the additional genes vif, vpr, vpx, vpu, tat, rev and nef for either vector production or for transduction of dividing and non-dividing cells. It has also been demonstrated that an EIAV minimal vector system can be constructed which does not require S2 for either vector production or for transduction of dividing and non-dividing cells. The deletion of additional genes is highly advantageous. Firstly, it permits vectors to be produced without the genes associated with disease in lentiviral (e.g. HIV) infections, improving the safety of the system. In particular, Tat, is associated with disease in HIV-1 infections. Secondly, the deletion of additional genes permits the vector to package more heterologous DNA. Thirdly, genes whose function is unknown, such as S2, may be omitted, thus reducing the risk of causing undesired effects. Examples of minimal lentiviral vectors are disclosed in WO-A-99/32646 and in WO-A-98/17815, as well as in U.S. Pat. No. 6,312,682, incorporated herein by reference.

Thus, preferably, the delivery system used in the invention is devoid of at least tat and S2 (if it is an EIAV vector system), and possibly also vif, vpr, vpx, vpu and nef. More preferably, the systems of the present invention are also devoid of rev. Rev was previously thought to be essential in some retroviral vector systems for efficient virus production. For example, in the case of HIV, it was thought that rev and RRE sequence should be included. However, it has been found that the requirement for rev and RRE can be reduced or eliminated by codon optimisation or by replacement with other functional equivalent systems such as the CTE (constitutive transport element) of MPMV (Mason Pfizer Monkey Virus). As expression of the codon optimised gag-pol is Rev-independent, RRE can be removed from the gag-pol expression cassette, thus removing any potential for recombination with any RRE contained on the vector genome. Thus, advantageously the viral genome may lack the Rev response element (RRE). An example of this may be found in Kotsopoulou et al. J. Virol, 74: 48399-4852.

In a preferred embodiment, the system used in the present invention is based on a so-called “minimal” system in which some or all of the additional genes have be removed.

Construction of the retroviral vector may also involve codon optimisation.

Codon optimisation has previously been described in WO99/41397. Different cells differ in their usage of particular codons. This “codon bias” corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available.

Many viruses, including HIV and other lentiviruses, use a large number of rare codons and by changing these to correspond to commonly used mammalian codons, increased expression of the packaging components in mammalian producer cells can be achieved. Codon usage tables are known in the art for mammalian cells, as well as for a variety of other organisms.

Codon optimisation has a number of other advantages. By virtue of alterations in their sequences, the nucleotide sequences encoding the packaging components of the viral particles required for assembly of viral particles in the producer cells/packaging cells have RNA instability sequences (INS) eliminated from them. At the same time, the amino acid sequence coding sequence for the packaging components is retained so that the viral components encoded by the sequences remain the same, or at least sufficiently similar that the function of the packaging components is not compromised. Codon optimisation also overcomes the Rev/RRE requirement for export, rendering optimised sequences Rev independent. Codon optimisation also reduces homologous recombination between different constructs within the vector system (for example between the regions of overlap in the gag-pol and env open reading frames). The overall effect of codon optimisation is therefore a notable increase in viral titre and improved safety.

In one embodiment of the present invention, the lentiviral vector is a self-inactivating vector. By way of example, self-inactivating retroviral vectors have been constructed by deleting the transcriptional enhancers or the enhancers and promoter in the U3 region of the 3′ LTR. After a round of vector reverse transcription and integration, these changes are copied into both the 5′ and the 3′ LTRs producing a transcriptionally inactive provirus (Yu et al. 1986 Proc Natl Acad Sci 83: 3194-3198; Dougherty and Temin 1987 Proc Natl Acad Sci 84:1197-1201; Hawley et al. 1987 Proc Natl Acad Sci 84: 2406-2410; Yee et al. 1987 Proc Natl Acad Sci 91: 9564-9568). However, any promoter(s) internal to the LTRs in such vectors will still be transcriptionally active. This strategy has been employed to eliminate effects of the enhancers and promoters in the viral LTRs on transcription from internally placed genes. Such effects include increased transcription (Jolly et al. 1983 Nucleic Acids Res 11: 1855-1872) or suppression of transcription (Emerman and Temin 1984 Cell 39: 449-467). This strategy can also be used to eliminate downstream transcription from the 3′ LTR into genomic DNA (Herman and Coffin 1987 Science 236: 845-848). This is of particular concern in human gene therapy where it is of critical importance to prevent the adventitious activation of an endogenous oncogene.

The adenovirus is a double-stranded, linear DNA virus that does not go through an RNA intermediate. There are over 50 different human serotypes of adenovirus divided into 6 subgroups based on the genetic sequence homology. The natural target of adenovirus is the respiratory and gastrointestinal epithelia, generally giving rise to only mild symptoms. Serotypes 2 and 5 (with 95% sequence homology) are most commonly used in adenoviral vector systems and are normally associated with upper respiratory tract infections in the young.

Viral gene expression can be divided into early (E) and late (L) phases. The late phase is defined by the onset of viral DNA replication. Adenovirus structural proteins are generally synthesised during the late phase. Following adenovirus infection, host cellular mRNA and protein synthesis is inhibited in cells infected with most serotypes. The adenovirus lytic cycle with adenovirus 2 and adenovirus 5 is very efficient and results in approximately 10, 000 virions per infected cell along with the synthesis of excess viral protein and DNA that is not incorporated into the virion. Early adenovirus transcription is a complicated sequence of interrelated biochemical events but it entails essentially the synthesis of viral RNAs prior to the onset of DNA replication.

The organisation of the adenovirus genome is similiar in all of the adenovirus groups and specific functions are generally positioned at identical locations for each serotype studied. Early cytoplasmic messenger RNAs are complementary to four defined, noncontiguous regions on the viral DNA. These regions are designated E1-E4. The early transcripts have been classified into an array of intermediate early (E1a), delayed early (E1b, E2a, E2b, E3 and E4), and intermediate regions.

The early genes are expressed about 6-8 hours after infection and are driven from 7 promoters in gene blocks E1-4.

Adenoviruses may be converted for use as vectors for gene transfer by deleting the E1 gene, which is important for the induction of the E2, E3 and E4 promoters. The E1-replication defective virus may be propagated in a cell line that provides the E1 polypeptides in trans, such as the human embryonic kidney cell line 293. A therapeutic gene or genes can be inserted by recombination in place of the E1 gene. Expression of the gene is driven from either the E1 promoter or a heterologous promoter.

Even more attenuated adenoviral vectors have been developed by deleting some or all of the E4 open reading frames (ORFs). However, certain second generation vectors appear not to give longer-term gene expression, even though the DNA seems to be maintained. Thus, it appears that the function of one or more of the E4 ORFs may be to enhance gene expression from at least certain viral promoters carried by the virus.

An alternative approach to making a more defective virus has been to “gut” the virus completely maintaining only the terminal repeats required for viral replication. The “gutted” or “gutless” viruses can be grown to high titres with a first generation helper virus in the 293 cell line but it has been difficult to separate the “gutted” vector from the helper virus.

The adenovirus provides advantages as a vector for identifying candidate modulating moieties over other gene therapy vector systems for the following reasons:

It is a double stranded DNA nonenveloped virus that is capable of in vivo and in vitro transduction of a broad range of cell types of human and non-human origin. These cells include respiratory airway epithelial cells, hepatocytes, muscle cells, cardiac myocytes, synoviocytes, primary mammary epithelial cells and post-mitotically terminally differentiated cells such as neurons.

Adenoviral vectors are also capable of transducing non dividing cells. This is very important for diseases, such as cystic fibrosis, in which the affected cells in the lung epithelium, have a slow turnover rate. In fact, several trials are underway utilising adenovirus-mediated transfer of cystic fibrosis transporter (CFTR) into the lungs of afflicted adult cystic fibrosis patients.

The expression of viral or foreign genes from the adenovirus genome does not require a replicating cell. Adenoviral vectors enter cells by receptor mediated endocytosis. Once inside the cell, adenovirus vectors rarely integrate into the host chromosome. Instead, it functions episomally (independently from the host genome) as a linear genome in the host nucleus. Hence the use of recombinant adenovirus alleviates the problems associated with random integration into the host genome.

Pox viral vectors may be used in accordance with aspects of the present invention, as large fragments of DNA are easily cloned into its genome and recombinant attenuated vaccinia variants have been described (Meyer, et al., 1991, J. Gen. Virol. 72: 1031-1038, Smith and Moss 1983 Gene, 25:21-28).

Examples of pox viral vectors include but are not limited to leporipoxvirus: Upton, et al. J. Virology 60:920 (1986) (shope fibroma virus); capripoxvirus: Gershon, et al. J. Gen. Virol. 70:525 (1989) (Kenya sheep-1); orthopoxvirus: Weir, et al. J. Virol 46:530 (1983) (vaccinia); Esposito, et al. Virology 135:561 (1984) (monkeypox and variola virus); Hruby, et al. PNAS, 80:3411 (1983) (vaccinia); Kilpatrick, et al Virology 143:399 (1985) (Yaba monkey tumour virus); avipoxvirus: Binns, et al. J. Gen. Virol 69:1275 (1988) (fowlpox); Boyle, et al. Virology 156:355 (1987) (fowlpox); Schnitzlein, et al. J. Virological Method, 20:341 (1988) (fowlpox, quailpox); entomopox (Lytvyn, et al. J. Gen. Virol 73:3235-3240 (1992)].

Poxvirus vectors are used extensively as expression vehicles for genes of interest in eukaryotic cells. Their ease of cloning and propagation in a variety of host cells has led, in particular, to the widespread use of poxvirus vectors for expression of foreign protein and as delivery vehicles for vaccine antigens (Moss, B. 1991, Science 252: 1662-7).

Pox viruses which may be used in accordance with aspects of the present invention include but are not limited to recombinant pox viral vectors such as fowl pox virus (FPV), entomopox virus, vaccinia virus such as NYVAC, canarypox virus, MVA or other non-replicating viral vector systems such as those described for example in WO 95/30018. Pox virus vectors have also been described where at least one immune evasion gene has been deleted (see WO 00/29428).

An nucleotide sequence or transgene used in a method of the present invention is inserted into any vector which is operably linked to a control sequence that is capable of providing for expression of the coding sequence by the host cell, i.e. the vector is an expression vector. The NOI produced by a host recombinant cell may be secreted or may be contained intracellularly depending on the nucleotide sequence and/or the vector used.

In a preferred embodiment, the vectors of the present invention are utilized to deliver one or more therapeutic genes either alone or in combination with other treatments or components of the treatment. For example, the vectors of the present invention may be used to deliver one or more transgenes useful in the treatment of the disorders listed in WO-A-98/05635. For ease of reference, part of that list is now provided: cancer, inflammation or inflammatory disease, dermatological disorders, fever, cardiovascular effects, haemorrhage, coagulation and acute phase response, cachexia, anorexia, acute infection, HIV infection, shock states, graft-versus-host reactions, autoimmune disease, reperfusion injury, meningitis, migraine and aspirin-dependent anti-thrombosis; tumour growth, invasion and spread, angiogenesis, metastases, malignant, ascites and malignant pleural effusion; cerebral ischaemia, ischaemic heart disease, osteoarthritis, rheumatoid arthritis, osteoporosis, asthma, multiple sclerosis, neurodegeneration, Alzheimer's disease, motor neuron diseases, neural regeneration for spinal cord accidents or other accidental injuries, atherosclerosis, stroke, vasculitis, Crohn's disease and ulcerative colitis; periodontitis, gingivitis; psoriasis, atopic dermatitis, chronic ulcers, epidermnolysis bullosa; corneal ulceration, retinopathy and surgical wound healing; rhinitis, allergic conjunctivitis, eczema, anaphylaxis; restenosis, congestive heart failure, endornetriosis, atherosclerosis or endosclerosis.

In addition, or in the alternative, the vectors of the present invention may be used to deliver one or more transgenes useful in the treatment of disorders listed in WO-A-98/07859. For ease of reference, part of that list is now provided: cytokine and cell proliferation/differentiation activity; immunosuppressant or immunostimulant activity (e.g. for treating immune deficiency, including infection with human immune deficiency virus; regulation of lymphocyte growth; treating cancer and many autoimmune diseases, and to prevent transplant rejection or induce tumour immunity); regulation of haematopoiesis, e.g. treatment of myeloid or lymphoid diseases; promoting growth of bone, cartilage, tendon, ligament and nerve tissue, e.g. for healing wounds, treatment of burns, ulcers and periodontal disease and neurodegeneration; inhibition or activation of follicle-stimulating hormone (modulation of fertility); chemotactic/chemokinetic activity (e.g. for mobilising specific cell types to sites of injury or infection); haemostatic and thrombolytic activity (e.g. for treating haemophilia and stroke); antiinflammatory activity (for treating e.g. septic shock or Crohn's disease); as antimicrobials; modulators of e.g. metabolism or behaviour; as analgesics; treating specific deficiency disorders; in treatment of e.g. psoriasis, in human or veterinary medicine.

In addition, or in the alternative, the vectors of the present invention may be used to deliver one or more transgenes useful in the treatment of disorders listed in WO-A-98/09985. For ease of reference, part of that list is now provided: macrophage inhibitory and/or T cell inhibitory activity and thus, anti-inflammatory activity; anti-immune activity, i.e. inhibitory effects against a cellular and/or humoral immune response, including a response not associated with inflammation; inhibit the ability of macrophages and T cells to adhere to extracellular matrix components and fibronectin, as well as up-regulated fas receptor expression in T cells; inhibit unwanted immune reaction and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex, HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyclitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery, bone marrow transplantation or other transplantation complications and/or side effects, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

Genes or Nucleotide Sequences of Interest (NOIs)

In the present invention, the term NOI (nucleotide sequence of interest) includes any suitable nucleotide sequence, which need not necessarily be a complete naturally occurring DNA or RNA sequence. Thus, the NOI can be, for example, a synthetic RNA/DNA sequence, a codon optimised RNA/DNA sequence, a recombinant RNA/DNA sequence (i.e. prepared by use of recombinant DNA techniques), a cDNA sequence or a partial genomic DNA sequence, including combinations thereof. The sequence need not be a coding region. If it is a coding region, it need not be an entire coding region. In addition, the RNA/DNA sequence can be in a sense orientation or in an anti-sense orientation. Preferably, it is in a sense orientation. Preferably, the sequence is, comprises, or is transcribed from cDNA.

The NOI(s), also referred to as “heterologous sequence(s)”, “heterologous gene(s)” or “transgene(s)”, may be any one or more of, for example, a selection gene(s), marker gene(s) and therapeutic gene(s).

The NOI may be a candidate gene which is of potential significance in a disease process. Thus the vector system of the present invention may, for example, be used for target validation purposes.

The NOI may have a therapeutic or diagnostic application. Suitable NOIs include, but are not limited to: sequences encoding enzymes, cytokines, chemokines, hormones, antibodies, anti-oxidant molecules, engineered immunoglobulin-like molecules, a single chain antibody, fusion proteins, immune co-stimulatory molecules, immunomodulatory molecules, anti-sense RNA, a transdominant negative mutant of a target protein, a toxin, a conditional toxin, an antigen, a tumour suppresser protein and growth factors, membrane proteins, vasoactive proteins and peptides, anti-viral proteins and ribozymes, and derivatives thereof (such as with an associated reporter group). The NOIs may s also encode pro-drug activating enzymes.

Preferably the NOI is useful in the treatment of a neurodegenerative disorder.

More preferably the NOI is useful in the treatment of Parkinson's disease.

The NOI may encode a growth factor such as GDNF, e.g., human GDNF or an analog, homolog, derivative or variant thereof, or an enzyme involved in dopamine synthesis or storage. For example, the enzyme may be one of the following: Tyrosine Hydroxylase, GTP-cyclohydrolase I and/or Aromatic Amino Acid Dopa Decarboxylase. The sequences of all three genes are available: Accession Nos. X05290, U19523 and M76180 respecively.

Alternatively the NOI may encode the vesicular monoamine transporter 2 (VMAT2, Accession number L23205.1). In a preferred embodiment the viral genome comprises an NOI encoding GDNF alone or in combination with Aromatic Amino Acid Dopa Decarboxylase and an NOI encoding VMAT 2. Such a genome may be used in the treatment of Parkinson's disease, in particular in conjunction with peripheral administration of L-DOPA.

Alternatively the NOI may encode a growth factor capable of blocking or inhibiting degeneration in the nigrostriatal system. An example of such a growth factor is a neurotrophic factor. For example the NOI may encode glial cell-line derived neurotrophic factor (GDNF), brain-derived neurotrophic factor (BDNF), nerve growth factor (NGF), persephin growth factor, artemin growth factor, or neurturin growth factor, cilliary neurotrophic factor (CNTF), neurotrophin-3 (NT-3), neurotrophin-4 (NT-4), pantropic neurotrophin, and other related or unrelated neurotrophic factors. WO99/14235; WO00/18799; U.S. Pat. No. 6,090,778; U.S. Pat. No. 5,834,914; WO97/08196; U.S. Pat. No. 6,090,778; U.S. Pat. No. 5,288,622; WO92/05254; U.S. Pat. No. 6,037,320; WO95/33829; Baumgartner, B J and Shine, H D, J. Neurosci. 17: 6504-11 (1997). In a preferred embodiment, a lentiviral vector comprises one or more of these NOls encoding neurotrophic factors.

Alternatively the NOI may encode a neuroprotective factor. In particular, the NOI(s) may encode molecules which prevent TH-positive neurons from dying or which stimulate regeneration and functional recovery in the damaged nigrostriatal system.

The NOI may encode all or part of the protein of interest (“POI”), or a mutant, homologue or variant thereof. For example, the NOI may encode a fragment of the POI which is capable of functioning in vivo in an analogous manner to the wild-type protein.

In a highly preferred embodiment, one of the NOIs comprises a truncated form of the TH gene, lacking the regulatory domain. Such an NOI avoids feed-back inhibition by dopamine which may limit expression of the full-length enzyme.

The term “mutant” includes POIs which include one or more amino acid variations from the wild-type sequence. For example, a mutant may comprise one or more amino acid additions, deletions or substitutions. A mutant may arise naturally, or may be created artificially (for example by site-directed mutagenesis).

Here, the term “homologue” means an entity having a certain homology with the NOI, or which encodes a protein having a degree of homology with the POI. Here, the term “homology” can be equated with “identity”.

In the present context, an homologous sequence is taken to include an amino acid sequence which may be at least 75, 85 or 90% identical, preferably at least 95 or 98% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

In the present context, an homologous sequence is taken to include a nucleotide sequence which may be at least 75, 85 or 90% identical, preferably at least 95 or 98% identical to the subject sequence. Typically, the homologues will comprise the same sequences that code for the active sites etc. as the subject sequence. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences. As used herein, % homology and % identity are interchangeable.

Percent homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Besifit package the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However, for some applications, it is preferred to use the GCG Bestfit program. A new tool, called BLAST 2 Sequences is also available for comparing protein and nucleotide sequence (see FEMS Microbiol Lett 1999 174(2): 247-50; FEMS Microbiol Lett 1999 177(1): 187-8 and tatiana@ncbi.nlm.nih.gov).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). For some applications, it is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

The sequences may also have deletions, insertions or substitutions of amino acid residues which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

The present invention also encompasses homologous substitution (substitution and replacement are both used herein to mean the interchange of an existing amino acid residue, with an alternative residue) may occur i.e. like-for-like substitution such as basic for basic, acidic for acidic, polar for polar etc. Non-homologous substitution may also occur i.e. from one class of residue to another.

Preferably the NOI encodes a single protein of interest (POI), e.g., GDNF such as human GDNF, or a mutant, homologue or variant thereof. In a highly preferred embodiment, the NOI does not encode a fusion protein. As used herein, the term “fusion protein” is used in its conventional sense to mean an entity which comprises two or more protein activities, joined together by a peptide bond to form a single chimeric protein. A fusion protein is encoded by a single polynucleotide driven by a single promoter.

In another preferred embodiment, the NOI comprises a small interfering or silencing RNA (siRNA). Post-transcriptional gene silencing (PTGS) mediated by double-stranded RNA (dsRNA) is a conserved cellular defence mechanism for controlling the expression of foreign genes. It is thought that the random integration of elements such as transposons or viruses causes the expression of dsRNA which activates sequence-specific degradation of homologous single-stranded mRNA or viral genomic RNA. The silencing effect is known as RNA interference (RNAi). The mechanism of RNAi involves the processing of long dsRNAs into duplexes of 21-25 nucleotide (nt) RNAs. These products are called siRNAs which are the sequence-specific mediators of mRNA degradation. In differentiated mammalian cells dsRNA >30 bp has been found to activate the interferon response leading to shut-down of protein synthesis and non-specific mRNA degradation. However this response can be bypassed by using 21 nt siRNA duplexes allowing gene function to be analysed in cultured mammalian cells.

In one embodiment an RNA polymerase III promoter, e.g., U6, whose activity is regulated by the presence of tetracycline may be used to regulate expression of the siRNA.

In another embodiment the NOI comprises a micro-RNA. Micro-RNAs are a very large group of small RNAs produced naturally in organisms, at least some of which regulate the expression of target genes. Founding members of the micro-RNA family are let-7 and lin-4. The let-7 gene encodes a small, highly conserved RNA species that regulates the expression of endogenous protein-coding genes during worm development. The active RNA species is transcribed initially as an ˜70 nt precursor, which is post-transcriptionally processed into a mature ˜21 nt form. Both let-7 and lin-4 are transcribed as hairpin RNA precursors which are processed to their mature forms by Dicer enzyme.

In a further embodiment the NOI comprises double-stranded interfering RNA in the form of a hairpin. The short hairpin may be expressed from a single promoter, e.g., U6. In an alternative embodiment an effective RNAi may be mediated by incorporating two promoters, e.g., U6 promoters, one expressing a region of sense and the other the reverse complement of the same sequence of the target. In a further embodiment effective or double-stranded interfering RNA may be mediated by using two opposing promoters to transcribe the sense and antisense regions of the target from the forward and complementary strands of the expression cassette.

In another embodiment the NOI may encode a short RNA which may act to redirect splicing (‘exon-skipping’) or polyadenylation or to inhibit translation.

Promoters/Enhancers

The NOI may be under the expression control of an expression regulatory element, usually a promoter or a promoter and enhancer. The enhancer and/or promoter may be preferentially active in a hypoxic or ischaemic or low glucose environment, such that the NOI is preferentially expressed in the particular tissues of interest, such as in the environment of a tumour, arthritic joint or other sites of ischaemia. Thus any significant biological effect or deleterious effect of the NOI on the individual being treated may be reduced or eliminated. The enhancer element or other elements conferring regulated expression may be present in multiple copies. Likewise, or in addition, the enhancer and/or promoter may be preferentially active in one or more specific cell types—such as any one or more of macrophages, endothelial cells or combinations thereof. Further examples include include respiratory airway epithelial cells, hepatocytes, muscle cells, cardiac myocytes, synoviocytes, primary mammary epithelial cells and post-mitotically terminally differentiated non-replicating cells such as macrophages and neurons. In particular, cells derived from the neuroepithelia such as neural and neuroglial cells are included.

The term “operably linked” means that the components described are in a relationship permitting them to function in their intended manner. A regulatory sequence “operably linked” to a nucleotide sequence of interest is ligated in such a way that expression of the nucleotide sequence is achieved under conditions compatible with the control sequences.

The term “promoter” is used in the normal sense of the art, e.g. an RNA polymerase binding site in the Jacob-Monod theory of gene expression.

The term “enhancer” includes a DNA sequence which binds to other protein components of the transcription initiation complex and thus facilitates the initiation of transcription directed by its associated promoter.

The promoter and enhancer of the transcription units encoding the secondary delivery system are preferably strongly active, or capable of being strongly induced, in the primary target cells under conditions for production of the secondary delivery system. The promoter and/or enhancer may be constitutively efficient, or may be tissue or temporally restricted in their activity. Examples of temporally restricted promoters/enhancers are those which are responsive to ischaemia and/or hypoxia, such as hypoxia response elements or the promoter/enhancer of a grp78 or a grp94 gene. One preferred promoter-enhancer combination is a human cytomegalovirus (hCMV) major immediate early (MIE). promoter/enhancer combination.

In one preferred embodiment the combined use of a strong constitutive promoter such as CMV, or house-keeping promoter such as PGK, and the Tet-regulation system may be used for control of gene expression. In addition to the Tet system other inducible systems include the met allothionein, hsp68, lacZ, and SV40 T antigen systems.

Gene Therapy

The present invention also provides a pharmaceutical composition for treating an individual by gene therapy, wherein the composition comprises a therapeutically effective amount of the vectors of the present invention comprising one or more deliverable therapeutic and/or diagnostic transgenes or a viral particle produced by or obtained from same. The pharmaceutical composition may be for human or animal usage. Typically, a physician will determine the actual dosage, based on available preclinical and clinical data, which will be most suitable for an individual subject and it may vary with the age, weight and response of the particular individual.

The composition may optionally comprise a pharmaceutically acceptable carrier, diluent, excipient or adjuvant. The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s), and other carrier agents that may aid or increase the viral entry into the target site (such as for example a lipid delivery system).

Where appropriate, the pharmaceutical compositions can be administered by any one or more of: inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intracavernosally, intravenously, intramuscularly, intracranially or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

The delivery of one or more therapeutic genes by a vector system according to the invention may be used alone or in combination with other treatments or components of the treatment. Diseases which may be treated include, but are not limited to: cancer, neurological diseases, inherited diseases, heart disease, stroke, arthritis, viral infections and diseases of the immune system.

Suitable therapeutic genes include but are not limited to those coding for tumour suppressor proteins, enzymes, pro-drug activating enzymes, immunomodulatory molecules, antibodies, engineered immunoglobulin-like molecules, fusion proteins, hormones, growth factors, membrane proteins, vasoactive proteins or peptides, cytokines, chemokines, anti-viral proteins, antisense RNA, ribozymes, and small interfering RNAs (siRNA).

Details on ribozymes may be found in “Molecular Biology and Biotechnology” (Ed. R A Meyers 1995 VCH Publishers Inc p 831-8320 and in “Retroviruses” (Ed. J M Coffin et al. 1997 Cold Spring Harbour Laboratory Press pp 683).

In a preferred embodiment of a method of treatment according to the invention, a gene encoding a pro-drug activating enzyme is delivered to a tumor or target tissue using the vector system of the invention and the individual is subsequently treated with an appropriate pro-drug. Examples of pro-drugs include etoposide phosphate (used with alkaline phosphatase Senter et al., 1988 Proc. Natl. Acad. Sci. 85: 4842-4846); 5-fluorocytosine (with Cytosine deaminase Mullen et al. 1994 Cancer Res. 54: 1503-1506); Doxorubicin-N-p-hydroxyphenoxyacetamide (with Penicillin-V-Amidase (Kerr et al. 1990 Cancer Immunol. Immunother. 31: 202-206); Para-N-bis(2-chloroethyl) aminobenzoyl glutamate (with Carboxypeptidase G2); Cephalosporin nitrogen mustard carba mates (with b-lactamase); SR4233 (with P450 Red uctase); Ganciclovir (with HSV thymidine kinase, Borrelli et al. 1988 Proc. Natl. Acad. Sci. 85: 7572-7576) mustard pro-drugs with nitroreductase (Friedlos et al. 1997 J Med Chem 40: 1270-1275) and Cyclophosphamide or Ifosfamide (with a cytochrome P450 Chen et al. 1996 Cancer Res 56: 1331-1340).

NOIs include but are not limited to: ApoE, Apo-SAA, BDNF, Cardiotrophin-1, EGF, ENA-78, Eotaxin, Eotaxin-2, Exodus-2, FGF-acidic, FGF-basic, fibroblast growth factor-10, FLT3 ligand, Fractalkine (CX3C), GDNF, G-CSF, GM-CSF, GF-β1, insulin, IFN-γ, IGF-I, IGF-II, IL-1α, IL-1β, IL-2, IL-3, IL-4, IL-5, IL-6, IL-7, IL-8 (72 a.a.), IL-8 (77 a.a.), IL-9, IL-10, IL-11, IL-12, IL-13, IL-15, IL-16, IL-17, IL-18 (IGIF), Inhibin α, Inhibin β, IP-10, keratinocyte growth factor-2 (KGF-2), KGF, Leptin, LIF, Lymphotactin, Mullerian inhibitory substance, monocyte colony inhibitory factor, monocyte attractant protein, M-CSF, MDC (67 a.a.), MDC (69 a.a.), MCP-1 (MCAF), MCP-2, MCP-3, MCP-4, MDC (67 a.a.), MDC (69 a.a.), MIG, MIP-1α, MIP-1β, MIP-3α, MIP-3β, MIP-4, myeloid progenitor inhibitor factor-1 (MPIF-1), NAP-2, Neurturin, Nerve growth factor, β-NGF, NT-3, NT-4, Oncostatin M, PDGF-AA, PDGF-AB, PDGF-BB, PF-4, RANTES, SDF1α, SDF1β, SCF, SCGF, stem cell factor (SCF), TARC, TGF-α, TGF-β, TGF-β2, TGF-β3, tumour necrosis factor (TNF), TNF-α, TNF-β, TNIL-1, TPO, VEGF, GCP-2, GRO/MGSA, GRO-β, GRO-γ and HCC1.

NOIs may also include marker genes (for example encoding β-galactosidase or green fluorescent protein) or genes whose products regulate the expression of other genes. In addition, NOIs may comprise sequences coding fusion protein partners in frame with a sequence encoding a protein of interest. Examples of fusion protein partners include the DNA binding or transcriptional activation domain of GAL4, a 6×His tag and β-galactosidase. It may also be desirable to add targeting sequences to target proteins encoding by NOIs to particular cell compartments or to secretory pathways. Such targeting sequences have been extensively characterised in the art.

Gene Therapy for Neurodegenerative Diseases

The vector system of the present invention is particularly useful for the treatment and/or prevention of neurodegenerative diseases as it is highly desirable to tightly regulate the administered therapeutic genes in the neurodegenerative setting. In this setting, it is preferred that target cells include cells derived from the neuroepithelia such as neural and neuroglial cells.

Diseases which may be treated include, but are not limited to: Parkinson's disease (PD); motor neuron disease, Huntington's disease and disorders of movement which are responsive to L-dopa, such as distonias.

In particular, the present invention is useful in treating and/or preventing PD. Treatment by gene therapy with vectors capable of delivering, for example, TH (tyrosine hydroxylase), or a mutant, variant or homologue thereof, GTP-CH1 (GTP-cyclohydrolase I), or a mutant, variant or homologue thereof, Aromatic Amino Acid Dopa Decarboxylase (or a mutant, variant or homologue thereof) in any order and optionally MDC (Aromatic Amino Acid Dopa Decarboxylase), or a mutant, variant or homologue thereof, or MDC and VMAT2 (vesicular monoamine transporter), is likely to be particularly useful for the late stages of PD patients which do not respond significantly to L-dopa treatment. Treatment using MDC or MDC and VMAT2, in combination with L-dopa administered peripherally may also be useful for late stage PD patients.

Treatment of neurodegenerative diseases may also be achieved using growth factor genes such as those growth factor genes capable of blocking or inhibiting degeneration in the nigrostriatal system. An example of such a growth factor is a neurotrophic factor. For example the gene may encode glial cell-line derived neurotrophic factor (GDNF), brain-derived neurotrophic factor (BDNF), nerve growth factor (NGF), persephin growth factor, artemin growth factor, neurturin growth factor, cilliary neurotrophic factor (CNTF), neurotrophin-3 (NT-3), neurotrophin-4 (NT-4), pantropic neurotrophin, or other related or unrelated neurotrophic factors. WO99/14235; WO00/1 8799; U.S. Pat. No. 6,090,778; U.S. Pat. No. 5,834,914; WO97/08196; U.S. Pat. No. 6,090,778; U.S. Pat. No. 5,288,622; WO92/05254; U.S. Pat. No. 6,037,320; WO95/33829; Baumgartner, B J and Shine, H D, J. Neurosci. 17: 6504-11 (1997). In a preferred embodiment, a lentiviral vector comprises one or more of these genes encoding neurotrophic factors.

In a particularly preferred embodiment, the cassette is bicistronic and comprises an NOI encoding TH, and an NOI encoding GDNF, and/or GTP-CH I, in either order. In another particularly preferred embodiment the cassette is bicistronic and comprises an NOI encoding GDNF and/or MDC and an NOI encoding VMAT2, in either order.

In another particularly preferred embodiment the cassette is tricistronic and comprises an NOI encoding GDNF and/or TH, an NOI encoding GTP-CH I and an NOI encoding AADC in any order.

The present invention also provides the use of an expression as defined herein in a pharmaceutical composition. The pharmaceutical composition may be used for treating an individual by gene therapy, wherein the composition comprises a therapeutically effective amount of a vector e.g., retroviral or lentiviral vector particle, according to the present invention.

The pharmaceutical composition may be used to treat a human or animal subject. Preferably the subject is a mammalian subject. More preferably the subject is a human. Typically, a physician will determine the actual dosage which will be most suitable for an individual subject and it will vary with the age, weight and response of the particular patient.

The composition may optionally comprise a pharmaceutically acceptable carrier, diluent, excipient or adjuvant. The choice of pharmaceutical carrier, excipient or diluent can be selected with regard to the intended route of administration and standard pharmaceutical practice. The pharmaceutical compositions may comprise as (or in addition to) the carrier, excipient or diluent, any suitable binder(s), lubricant(s), suspending agent(s), coating agent(s), solubilising agent(s), and other carrier agents that may aid or increase the viral entry into the target site (such as for example a lipid delivery system).

Where appropriate, the pharmaceutical compositions can be administered by any one or more of: inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intracavernosally, intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

Preferably the viral vector particles of the present invention are administered by injection into the caudate putamen or intracranially, e.g., to striatum and/or substantia nigra.

The invention also comprehends treating or preventing diseases or maladies or conditions or symptoms thereof.

The retroviral vector genome and vector particles of the present invention are particularly useful for the treatment and/or prevention of neurodegenerative diseases. Diseases which may be treated include, but are not limited to: Parkinson's disease; motor neuron disease, Huntington's disease and disorders of movement which are responsive to L-dopa, such as distonias. In particular, the present invention is useful in treating and/or preventing Parkinson's disease.

Treatment by gene therapy with vectors capable of delivering, for example, GDNF alone or in combination with any or all of TH, GTP-CH1 and optionally MDC or MDC and VMAT2, is likely to be particularly useful for the late stages of PD patients which do not respond significantly to L-dopa treatment. Treatment using MDC or MDC and VMAT2, in combination with L-dopa administered peripherally may also be useful for late stage PD patients.

The present invention will now be further described by way of the following non-limiting examples, provided for illustrative purposes only.

### EXAMPLES

### Example 1

### Regulated Expression of Glial Cell Derived Growth Factor in an EIAV-Based Vector System

Glial-cell derived growth factor (GDNF) has been shown to have potent effects in preventing death of dopaminergic neurons in the substantia nigra in animal models of Parkinsons disease. This observation has led to the idea of human gene therapy using an expression cassette for GDNF, delivered to cells of the substantia nigra (e.g. U.S. Ser. No. 10/008,610). However it is generally agreed that unregulated, constitutive expression of GDNF in a gene therapy application is undesirable due to the potential for inappropriate growth of neurons. In this example we demonstrate a means of controlling expression of GDNF when delivered by an EIAV vector system. The system described here comprises a switch by which GDNF expression is turned off by addition of the drug diflucan also known as Fluconazole, (Kaufman D. et al., N. Engl J Med; 345:1660-1666, 2001; Schutze G. E. et al. N Engl J Med; 330:1759-1760, 1994).

The basic structure and mode of operation of the system is as follows. The control mechanism consists of two aptamers and is placed within the 5′ untranslated region of the GDNF open reading frame. The structure of the mechanism is as follows: Aptamer A (selected for binding to diflucan)—a spacer sequence—aptamer B (selected for binding to the aptamer A—diflucan combination). In the absence of diflucan, translation of GDNF takes place as a result of a cap-dependent/ribosome scanning translational mechanism however in the presence of the drug, scanning is inhibited by formation of a tripartite complex between aptamer A, diflucan and aptamer B. The blocking effect of the combination of these aptamers is an improvement over the blocking effect observed for single aptamers, or for tandem arrays of a single aptamer such as those described previously, due to the greater stability of the tripartite complex.

The basic vector into which the control system is introduced is pONY8-GDNF from which constitutive expression of GDNF is driven by an internal human cytomegalovirus immediate early promoter. pONY8-GDNF was derived from a modified form of pONY8G (WO00/229065; Martin-Rendon E, et al. Mol Ther. 5:566-570, 2002). The modification removes the BstXI site by cutting with BstXI, blunting the ends using T4 DNA polymerase and then religating, and the modified form of the vector termed pONY8Gx. The GDNF ORF is inserted between the SacII and NotI sites of pONY8Gx to create pONY8-GDNF. Alternatively, the GDNF ORF can be inserted between the SacII and NotI sites of an EIAV vector backbone such as pONY8.1G.

In order to allow insertion of the aptamers pONY8-GDNF is modified to include a linker containing two SfiI sites with different overhangs and two BstXI sites with different overhangs, the linker being located in the 5′UTR of the GDNF gene. The use of different overhangs allows directional cloning of the aptamers derived from the selection process. This modified vector plasmid is termed pONY8-Sfi/Bst-GDNF.

The RNA aptamer library is generated from a short DNA template with the following structure, using a T7 RNA polymerase in vitro transcription reaction:
T7-Promoter-18 bp 5′ of BstXI-SfiI-linker A N40 to 100-linker B-18 bp 3′ of SfiI-BstXI

The 18 base pair sequences to the 5′ and 3′ BstXI sites are present to act as targets for primers used in RT-PCR reactions to amplify aptamers during the selection process. The sequences of the SfiI and BstXI sites are the same as those in pONY8-Sfi/Bst-GDNF and also in the same 5′-3′ order to allow directional cloning. Linker A and B are 15 base sequences designed to have minimal secondary structure and are included to ensure that the aptamer library is insulated from the effects of adjacent sequences after it is inserted into the EIAV vector, and thus should have ligand binding properties the same as those displayed during the selection process. The influence of flanking sequences on aptamer activity has been noted previously (Martell et al. Mol Ther, Vol. 6:30). The sequences of Linker A and B are AGTTCACGATCCAAGCTG (SEQ ID NO:1) and ATCCTGMGCCACGAGTT (SEQ ID NO:2), respectively. The linkers are designed to not contain the trinucleotide ATG as this would cause translation initiation.

The template is assembled by PCR using oligonucleotide primers. The PCR is performed in the presence of DMSO or other commercial reagents that allow amplification of DNA with secondary structure.

Following assembly of the template by PCR it is purified by gel electrophoresis and then used in in vitro reactions using a commercial T7 RNA synthesis kit. The RNA library generated is gel purified using a polyacrylamide gel electrophorsesis and then eluted overnight in binding buffer (50 mM Tris ph 8.3, 250 mM KCl, 2 mM MgCl₂, 1 mM DTT).

In order to increase the efficiency of selection studies it is necessary to first remove molecules which have a high affinity for the column matrix. This preselection is achieved as follows. A proportion of the library is denatured at 70° C. for 3 min and allowed to cool to room temperature. This pool of RNA molecules is then passed through 0.5 ml agarose column which is equilibrated using binding buffer. Unbound RNA is eluted with 2 volumes of binding buffer. The eluted pool is then applied to 0.5 ml of the agarose affinity matrix linked to diflucan. Linkage is achieved using cyanogen bromide activated agarose. A matrix with slightly altered binding properties can be made by linking Diflucan via a linker to the column matrix. The column is subsequently washed with 20 to 50 ml of binding buffer and eluted with binding buffer containing 10 mM diflucan. The eluted RNA is precipitated and reverse transcribed into cDNA using the primer 2. The cDNA is amplified by PCR using flanking primers 1 and 2. This enriched pool is purified and reverse transcribed as described above and then used for an additional round of selection. Several rounds of selection which result in enrichment of aptamers with high affinity can be performed.

Enrichment for specific RNA aptamers results in a gradual increase in the relative amount of aptamers bound to the selection column. In later rounds of selection a larger fraction of the RNA pool is retained on the column because of its affinity to the substrate it is selected for. Enrichment is assessed by keeping aliquots of RNA before the column and from the eluted, enriched RNA. This RNA is subjected to quantitative RT-PCR using primers 1 and 2 and cybergreen for analysis by realtime PCR. If the protocol results in an enrichment of specific RNA's the difference between the threshold value (Ct) obtained from material before and after selection will decrease. The decrease in Ct ratios are used to decide when PCR products from the selected pools are then cloned and sequenced. Effective selection takes place in 5 to 20 rounds of selection.

Aptamer-Diflucan binding constants (Kd) for individual aptamers derived via the described selection scheme are estimated by gel electrophoretic mobility shift assays (EMSA's). The inserted sequences of plasmid constructs including the T7 promoter region is amplified by PCR and transcribed using commercial T7 RNA polymerase in the presence of trace amount of radioactive ATP, and then purified. A fixed amount of aptamer RNA (about 10 nmoles) is mixed with various amount of diflucan made up in binding buffer, heated to 65° C. and then cooled to room temperature. Binding of diflucan to the RNA results in a altered electrophoretic mobililty which is detectable in EMSA's carried out using a 6% polyacrylamide gel. Quantitation of free and complexed RNA aptamer is performed by autoradiography and binding constants are determined assuming a single-site binding model. Alternatively, the affinity of the aptamer to diflucan can be estimated by measuring the concentration of diflucan necessary to elute the aptamer RNA from the column.

It would be expected that the aptamer A-diflucan combination would be able to modulate expression albeit not very efficiently. This could be demonstrate by introducing the DNA form of the aptamer into the SfiI site of the vector genome plasmid pONY8-Sfi/Bst-GDNF or its analogue pONY8-Sfi/Bst-Z, derived from pONY8Z. Vector particles are produced from the these vector genome plasmids by co-transfection of 293T cells with Gag/Pol expression plasmid, pEsynGP, an expression plasmid for EIAV Rev protein, pEsynRev and an expression plasmid for the G protein of vesicular stomatitis virus, pRV67. The titre of the resulting vector preparation is determined using a quantitative RT-PCR based assay for the the detection of RNA's containing the EIAV packaging signal. The efficiency of regulation mediated by diflucan is determined initially in vitro using 293 and D17 cell lines and primary neuron cultures. Expression levels of GDNF or β-galactosidase are compared with that of the parental vectors derived from pONY8Z and pONY8-GDNF using quantitative assays. For example, GDNF can be detected using an ELISA; R&D Elisa Kit DY212.

Since the Diflucan used has a MW of only 306 D, the binding affinities (Kd) that can be achieved are very likely to be in the range of 50 to 500 uM for a single aptamer. In order to reduce the off rate and therefore increase the affinity of Diflucan, a second aptamer B with high affinity for the Diflucan-aptamer A complex is generated. This second aptamer B is selected from a pool generated in a similar manner to that previously described above.

As a preliminary step, the RNA will be negatively selected on 0.5 ml of Agarose-aptamer A to remove RNA's binding to the column matrix or to aptamer A in the absence of Diflucan. The eluted RNA pool is then used in a positive selection procedure using a column containing the aptamer A-Diflucan complex. After the binding step the column is washed with 10 to 20 volumes of binding buffer containing 50 uM Diflucan to maintain the tertiary complex. At this concentration any aptamer B with a higher affinity for free diflucan than for the aptamer A-diflucan is eluted. The remaining bound RNA is eluted with free aptamer A-diflucan complex, concentrated by precipitation, and amplified by RT-PCR for the next round of selection. As for selection of aptamer A, the enrichment of RNA is followed by quantitative RT-PCR for RNA pre column and post elution for 10 to 20 rounds of selection.

For functional analysis the cDNA form of aptamer B is into pONY8-Sfi/Bst-GDNF-aptA vector or the pONY8Z derivative using the BstXI sites. Similar tests for control of gene expression as those described above are then carried out. Aptamer B is introduced adjacent to Aptamer A in the example described here; however it can also be introduced into the 3′UTR of the gene for which expression is to be regulated.

### Example 2

### Treatment of MPTP (1-Methyl-4-Phenyl-1,2,3,6 Tetrahydropyridine)-Ablated Monkeys with a Lentiviral Vector Expressing GDNF Under the Control of Diflucan and Comparison with Unregulated Vector

These experiments show that if diflucan (fluconazole) is administered continuously (3-12 mg/kg×day) to an animal previously treated with a lentiviral vector carrying the GDNF ORF (Lenti-GDNF) as described in Example 1, that GDNF expression is switched off by the presence of drug, no therapeutic effect is seen in Parkinson's model monkeys, and that no significant levels of GDNF can be measured in the brains of diflucan treated animals compared to animals where diflucan is omitted. Vector is prepared as described in Example 1 and concentrated to a minimum of 10⁹ transforming units/ml in formulation buffer.

Twenty young adult rhesus are initially trained 3 days per week until asymptotic performance is achieved on a hand-reach task in which the time to pick up food treats out of recessed wells is measured (M. E. Emborg, et al., J. Comp. Neurol. 401, 253 (1998); J. H. Kordower, Cell Transplant. 4, 155 (1995)). Each experimental day, monkeys receive 10 trials per hand. Once per week, monkeys are also evaluated on a modified parkinsonian clinical rating scale (CRS) to obtain a measurement of baseline performance. All monkeys then receive an injection of 3 mg MPTP-HCl into the right carotid artery, initiating a Parkinsonian state. One week later, monkeys are evaluated on the CRS. Only monkeys displaying severe hemi-Parkinsonism with the classic crooked arm posture and dragging leg on the left side continue in the study. Normally, monkeys with this behavioral phenotype display the most severe lesions neuroanatomically and do not display spontaneous recovery behavior (Kordower op.cit).

On the basis of CRS scores, monkeys are matched into three groups of five monkeys, which receive on that day: lentiviral vector expressing-b-galactosidase (Lenti-βgal) (group 1); Lenti-GDNF plus continuous treatment with diflucan (group 2); or Lenti-GDNF treatment alone (group 3). Using magnetic resonance imaging is (MRI) guidance, each monkey receives six stereotactic injections of Lenti-βgal or Lenti-GDNF bilaterally into the caudate nucleus, putamen, and substantia nigra. Injections are made into the head of the caudate nucleus (10 μl), body of the caudate nucleus (5 μl), anterior putamen (10 μl), commissural putamen (10 μl), postcommissural putamen (5 μl), and substantia nigra (5 μl). Injections are made using a 10 μl Hamilton syringe (31 gauge), connected to a pump, at a rate of 0.5 μl/minute (min). During the injection, the needle is raised 1 to 2 mm to better disperse the vector through the intended target tissue. The needle is left in place for 3 min after injection of vector has been completed to minimize back diffusion of the vector. The left side is injected 6 weeks before the right hemisphere. All experimentation is performed in accordance with NIH guidelines and institutional animal care approval. Level II Biosafety procedures are used.

One week following lentiviral vector treatment, monkeys begin retesting on the hand-reach task three times per week for 3 weeks per month. Testing is performed during weeks 2, 3 and 4 of the first month after surgery to allow the animals time to recover from surgery, and in weeks 1,2 and 3 of months 2 and 3. For statistical analyses, the times for an individual week are combined into a single score. During the weeks of hand-reach testing, monkeys are also scored once per week on the CRS. Three months after lentiviral vector treatment, monkeys receive a Fluoro-Dopa (FD) PET scan. The monkeys are sacrificed 24 to 48 hours later, and the tissues, processed for histology.

Before MPTP treatment, all young adult monkeys score 0 on the CRS. After MPTP, but before injection with vector, monkeys in all groups average about 10 on the CRS. After vector treatment, significant differences in CRS scores are seen between the groups (Kolmogorov-Smirnov test). CRS scores of monkeys receiving Lenti-βgal or Lenti-GDNF plus diflucan do not change over the 3-month period after treatment. In contrast, CRS scores of monkeys receiving Lenti-GDNF alone significantly diminish during the 3-month period after treatment with scores beginning to decrease in the first month after Lenti-GDNF treatment. Lenti-GDNF-treated animals also improved performance on the operant handreach task. Under the conditions before MPTP administration, animals in all three groups perform this task with similar speed. For the “unaffected” right hand, no differences in motor function are discerned for any group relative to performance before MPTP administration or to each other. In contrast, performance with the left hand is significantly improved in Lenti-GDNF-treated animals relative to animals also receiving Lenti-GDNF plus diflucan or animals receiving Lenti-βgal alone. The performance of all Lenti-βgal-treated and all Lenti-GDNF plus diflucan treated animals are severely impaired, with monkeys often not performing at all, or requiring more than the maximally allowed 30 seconds (s) to complete the test. In contrast, most of the Lenti-GDNF monkeys perform the task with the left hand at near-normal levels.

Prior to sacrifice, all monkeys undergo FD PET scans. All procedures follow an overnight fast. After sedation with ketamine (10 to 15 mg/kg), the animal is intubated, and femoral angiocatheters are placed for tracer injection and blood sampling. Anesthesia is then maintained by 1 to 2% isofluorane for the remainder of the procedure. Carbidopa (2 to 3 mg/kg IV) is administered 30 min before the FD study. The animal is placed in a stereotaxic head holder constructed of materials compatible with PET scanning, and a transmission scan is acquired for correction of the emission data for attenuation. FD (185 MBq) is administered over 30 s and a 90 min three-dimensional dynamic emission scan started. The scan includes 22 frames with durations increasing from 1 min initially to 5 min at the end. The bed is moved cyclically by the interplane distance between each pair of 5-min scans to give a net coronal sampling interval of 2.125 mm. Regions of interest (ROI) were placed located within the caudate nucleus, putamen and occipital cortex in individual morphometric MR images coregistered with the FD image data. Cortical time courses were used as input functions to generate functional maps of the uptake rate constant, Ki, by the modified graphical method [C. S. Patlak, R. G. Blasberg, J. Cereb. Blood Flow Metab. 5, 584 (1985)]. Striatal ROI's were transferred to the functional maps, and the Ki values were evaluated as the ROI means for each structure.

Qualitatively, all Lenti-βgal-treated and all Lenti-GDNF plus diflucan treated monkeys display pronounced FD uptake in the left striatum and a comprehensive loss of FD uptake on the right side. In contrast, at least some of the Lenti-GDNF-treated animals display robust and symmetrical FD uptake on both sides, and most show reduced FD uptake on the right side, but with Ki values 50 to 100% greater than Lenti-βgal controls. Quantitatively, no differences in FD uptake is observed between groups within the left striatum. There is a significant increase in FD uptake in Lenti-GDNF-treated animals in the right striatum relative to Lenti-βgal-treated animals.

After euthanasia, all monkeys are perfused with saline. The brain is removed, immersed in ice-cold saline for 10 min, and sliced on a monkey brain slicer. Slabs through the head of the caudate and putamen are punched bilaterally with a 1 mm brain punch. These punches are processed for HPLC for determination of catecholamine content. Brain punches are also homogenized in 150:1 buffer I [0.1M tris-buffered saline, pH 8.1, containing 1 mM EDTA, 1% aprotinin, 10 mg/ml leupeptin, 14 mg/ml pepstatin, 4 mM phenylmethylsulfonyl fluoride (PMSF)] for 30 sec in the ice slurry. An equal amount of buffer II (0.1 M tris-buffered saline, pH 8.1, containing 1 mM EDTA, 1% aprotinin, 10 g/ml leupeptin, 14 μg/ml pepstatin, 4 mM PMSF, and 0.5% NP-40) is then added. The tubes are shaken for 2 hours. The supernatant is collected for ELISA and protein measurements. The ELISA reaction was completed in 96-well plate (Dynatech, Chantilly, Va.) according to the ELISA manufacturer's instructions (GDNF Eₘₐₓ ImmunoAssay Systems Kit G3520, Promega, Madison, Wis.). The optical densities are recorded in ELISA plate reader (at 450 nm wave length; Dynatech). Some lysates are diluted to ensure all the optical densities are within the standard curve. The concentrations of GDNF are calculated against six-point standard curve and then adjusted to picograms of GDNF per milligram of total protein. The total protein in each tissue lysate is measured using Bio-Rad protein assay kit (Bio-Rad, Richmond, Calif.).

The tissue slabs are immersed in Zamboni's fixative. Stereological counts and volumes of TH-immunoreactive neurons are performed with NeuroZoom software using the optical dissector method for cell counting and the nucleator method for measuring neuronal volume. The TH riboprobe is prepared as previously described. The cDNA is transcribed in the presence of biotin-14-CTP (Gibco BRL/Life Technologies, Rockville, Md.), 1 mg Pvu I-linearized pBS-TH39, 5 mM DTT, 50 U RNasin, 4 U T3RNA polymerase, 0.5 mM CTP, and 0.25 mM of ATP, GTP, and UTP. Tissue is processed for immunohistochemistry by the ABC method using this probe as the primary antibody. Optical density measurements are performed using NIH Image.

A strong GDNF-immunoreactive signal is seen in the caudate nucleus,putamen, and substantia nigra of all Lenti-GDNF-treated, but little or no signal is seen in the Lenti-βgal and Lenti-GDNF plus diflucan treated animals. GDNF immunohistochemistry is performed with a commercially available antibody (R&D Systems, Minneapolis, Minn.; 1:250), using the ABC method and nickel intensification. Deletion or substitution for the primary antibody serve as a control. Under control conditions, no staining is observed.

The Lenti-βgal-treated and Lenti-GDNF plus diflucan treated monkeys display a loss of TH immunoreactivity within the striatum on the side ipsilateral to the MPTP injection. In contrast, Lenti-GDNF-treated monkeys display enhanced striatal TH immunoreactivity relative to Lenti-βgal controls. However, there is variability in the degree of striatal TH immunoreactivity in Lenti-GDNF-treated animals and that variability is associated with the degree of functional recovery seen on the hand-reach task.

### Example 3

### Gene Regulation by Aptazymes—Aptamer-Ribozyme Hybrids

In the control systems described in Examples 1 and 2 the presumed mechanism of expression control is via interference of translation by impeding the progress of the ribosome as it scans towards the start codon. An alternative way of inhibiting translation is to destabilize the mRNA. In this example we demonstrate a ribozyme which is activated by interaction of an aptamer with its ligand. This new moiety is termed an aptazyme. Activation leads to cleavage of a cognate sequence within the mRNA, for example just upstream of the polyA sequence, leading to downregulation of expression. This system has the advantage over the simple ‘blocking’ aptamers of Example 1 and 2 that cleavage of the RNA is irreversible and may also require a less stable aptamer-ligand interaction in view of the relatively short life-time required for substrate cleavage to occur. In addition, there is the possibility of trans activity leading to enhanced efficacy.

The aptazyme is selected from a library of RNA molecules for its function to self cleave only after addition of a specific ligand and thereby remove the poly(A) tail.

Similar to examples 1 and 2 the process starts with insertion of one or 2 unique restriction sites into the 3′UTR of the gene to be regulated. In practice such a system could also be used in a 5′UTR position however these regions of mRNA are usually more complex due to the sequence constraints required for efficient translation. Because the selection process might select for aptamers which include sequences recognized by restriction enzymes when they are converted to the DNA form, the restriction site is preferably an enzyme with the more rare 8 bp recognition sequence.

Several methods of selection are possible

A) Coselection of a ribozyme/aptamer combination which can be regulated by ligand addition. Essentially direct selection of a regulatable ribozyme.

A T7 RNA polymerase template library for expression of RNA molecules of the following structure is generated as described in example 1, and has the following configuration:
T7 promoter—unique restriction site 1-30mer linker sequence—the raptazyme library (40-100mers)—a linker sequence—ribozyme cleavage site (GAUGGUUACUCCAAGCG; SEQ ID NO:3)—unique restriction site 2—polyA sequence.

The ribozyme cleavage site incorporates sites preferable for hammerhead ribozyme which cleave after UH (H is A, U, or C) and deoxyribozymes which prefer to cleave between unpaired purine and pyrimidines dinucleotides. The sequence shown provides a sampling of all 16 possible dinucleotides and is taken from Tang and Breaker, PNAS 97:5784.

The purpose of the polyA sequence is to provide a means of anchoring the raptazyme to the column matrix, which in this case is agarose-poly dT beads.

In the first step of selection the aptazyme library is applied to the column and then any ribozymes are activated by addition of buffer containing Mg2+. This process results in loss of raptazymes which have cleavage activity in the absence of ligand (e.g., diflucan) aptazymes which are active in the presence of ligand are then selected by washing the column with Mg2+-containing buffer supplemented with ligand. This RNA is amplified and subject to the next round of selection.

B) Selection of a raptazyme which includes an aptamer which has already been selected for interaction with a given ligand.

In this strategy the aptazyme library component of the T7 RNA polymerase template is composed of an aptamer specific for a ligand flanked by random nucleotides (10 to 30mers). Negative and positive selections are carried out as described for A).

C) Selection of a aptazyme which includes a predefined ribozyme and then insertion random nucleotides (20 to 100mers) in a loop of the ribozyme.

The library of variants so-formed will contain species capable of binding ligand and these interactions may allow regulation of ribozyme activity. Negative and positive selections are carried out as described for A)

D) Replacement of the helix II of a ribozyme with an aptamer linked by a randomized bridge sequence.

This strategy takes advantage of the modular nature of ribozymes and aptamers as demonstrated by Soukup and Breaker (PNAS 96:3584). The randomized bridge sequence allows for selection of a suitable context for the aptamer to regulate the ribozyme. Negative and positive selections are carried out as described for A).

For some applications it might be desirable to have the gene of interest expressed at a low level and inducibly upregulated by a drug. This is achieved by changing the selection process of the aptazyme. First, selection will be for aptazymes which are not cleaved in the presence of the inhibitor. The second step is to positively select for self-cleaving aptazymes after the ligand is washed out. This might involve denaturing and renaturing of the aptazyme if the ligand is bound with a high affinity that won't allow dissociation of the drug from the aptazyme.

### Example 4

### Construction of an Aav Vector Plasmid Which Contains an Ires-Aptamer Control System for Regulated Expression of Erythropoietin (Epo)

The system described below contains two different aptamers and is configured so that expression of Epo can be turned on by addition of tetracycline (tet), according to the following rationale. In the absence of tet, Aptamer B binds to the upstream aptamer A, thus preventing usage of an intervening IRES hence a low level of translation. This interaction may be specific to the secondary structure of aptamer A (Cho B, Taylor D C, Nicholas H B Jr, Schmidt F J “Interacting RNA species identified by combinatorial selection.” Bioorg Med Chem 1997; June; 5(6):1107-13) or be due to other interacti0ons such as conventional nucleic acid base pairing.

However in the presence of tet the aptamer A-B complex is interrupted due to binding of tet to aptamer A, thereby releasing the IRES from the inbitory configuration, resulting in increased levels of translation. Aptamer B should no affinity for tet so that there is no reduction of translation due to the interaction of tet with aptamer B.

The operation of the inducible system is demonstrated in the context of the MV vector MV-CMV-EPO. The MV-CMV-EPO vector expresses murine EPO (eEPO) constitutively from a CMV promoter and has been previously described. Briefly, between the ITR's are located (in the negative sense) SV40polyA signal—mEPO-intron—CMV immediate early promoter and (in the positive sense) a fragment of the B-galactosidase gene which acts as a “stuffer” to ensure a correct size of the DNA for efficient packaging. The control system consists of 2 aptamers, A, to the 5′ side, and B, to the 3′ side, of an internal ribosome entry, derived from poliovirus, and located in the 5′UTR of the murine erythropoetin (mEPO).

Aptamer A is selected from the library of aptamers described in example 1, to bind to tet with high affinity as illustrated in example 1. The second aptamer B is selected from a pool of aptamers in a two-step process, first by negative selection to a complex of agarose-aptamerA-tet and secondly by positive selection of aptamers from the new pool with Agarose-aptamer A over 10 to 20 rounds of selection. Aptamers binding to the column are eluted with addition of 1 um to 10 mM of aptamer A or by heat disassociaton.

The cDNA form of Aptamer A is obtained by PCR amplification using primers which introduce at the 5′ end, SalI and BglII sites and at the 3′ end, a SalI site. This PCR product is then digested with SalI and ligated into pBL-EP prepared for ligation by digestion with SalI. pBL-EP is a plasmid vector which contains both the encaphalomyocarditis virus and poliovirus IRESes, connected by multiple cloning sites. The product of the ligation was called pBL-EP-aptA. Aptamer B was obtained by PCR amplification using primers which introduced 5′-flanking MluI and 3′-flanking, NcoI and XhoI sites, respectively. This PCR product was then digested with MluI and XhoI and ligated into pBL-EP-aptA prepared for ligation by digestion with the same enzymes. The final product was termed pBL-EP-aptAB. These manipulations place aptamers A and B upstream and downstream of the poliovirus IRES in such a way that the aptamer-IRES cassette can be excised by digestion with BglII and NcoI.

The aptamer A-IRES-aptamer B complex is built into the AAV-CMV-Epo vector in a series of steps. In the first step the SpeI-XhoI fragment which spans the CMV promoter, the intron and the mEPO ORF from MV-CMV-Epo is ligated into pBS II KS+ which is prepared for ligation by digestion with the same enzymes. This plasmid is termed pBS.mEPO. pBS.mEPO is then modified to create a BglII and NcoI site at the start of the murine EPO open reading frame. This is achieved using “overlapping” PCR using pBS.mEPO as template and primers as follows:

(SEQ ID NO:4)

5′EPO: TAACCCCGCCCCGTTGACGC
and

(SEQ ID NO:5)

3′EPO GGCCTGTTCTTCCACCTCCATTCT;

(SEQ ID NO:6)

intEPO pos:
CCCATATGGATCCAGCTAGGCGCagatctttaaCACCATGGGGGTGCCCG
AACGTCCCAC

(SEQ ID NO:7)

intEPO neg:
GTGGGACGTTCGGGCACCCCCATGGTGttaaagatctGCGCCTAGCTGGA
TCCATATGGG.

In the first phase of the PCR reaction 5′EPO and intEPO neg, and 3′EPO and intEPO pos are used to create, respectively 1) a fragment which spans the unique HindIII fragment of the plasmid and the start codon of mEPO and 2) a fragment which spans from the start of mEPO to 3′ of the unique PpuMI site present in the mEPO gene. These two fragments were then used in a new “overlapping” PCR reaction using in the first 10 cycles, no PCR primers, and then for the remaining 25 cycles primers 5′EPO and 3′EPO.

The 690 bp fragment of DNA obtained from the reaction is digested with HindIII and PpuMI and then ligated into pBS.mEPO prepared for ligation with the same enzymes, to create pBS.mEPO-BglII/NcoI. The BglII and NcoI sites are used to accept a fragment of DNA which contained aptamer A—PV-IRES—aptamer B which is released from pBL-EP-aptAB by digestion with BglII and NcoI. In the final step the SpeI-XhoI fragment is transferred back to the MV-CMV-Epo backbone.

The regulatory properties of the system are analysed using the mouse model system described in Binley K et al. (Blood, 100: 2406-2413,2002) and in U.S. Ser. No. 10/066,218, in which regulated expression of Epo was achieved using a hypoxia regulated transcriptional element. However, in this instant exemplification, expression of Epo is controlled by external administration of varying concentrations of tetracycline.

The invention is further described by the following numbered paragraphs:
1. An expression vector comprising at least one nucleotide sequence of interest (NOI) and at least one aptamer sequence, wherein the at least one aptamer sequence is operably linked to the NOI, for use in medicine.
2. A pharmaceutical composition comprising an expression vector comprising at least one nucleotide sequence of interest (NOI) and at least one aptamer sequence, wherein the at least one aptamer sequence is operably linked to the NOI, together with a pharmaceutically acceptable carrier, diluent, excipient or adjuvant.
3. Use of an expression vector comprising at least one nucleotide sequence of interest (NOI) and at least one aptamer sequence, wherein the at least one aptamer sequence is operably linked to the NOI, for the preparation of a medicament for regulating gene expression in an animal.

Having thus described in detail preferred embodiments of the present invention, it is to be understood that the invention defined by the appended claims is not to be limited to particular details set forth in the above description, as many apparent variations thereof are possible without departing from the spirit or scope of the present invention. Modifications and variations of the method and apparatuses described herein will be obvious to those skilled in the art, and are intended to be encompassed by the following claims.

1. An expression vector comprising at least one nucleotide sequence of interest (NOI) and at least one aptamer sequence, wherein the at least one aptamer sequence is operably linked to the NOI.

2. The expression vector of claim 1, wherein the at least one aptamer sequence is incorporated in the 5′ untranslated region (UTR) of the NOI.

3. The expression vector of claim 1, wherein the at least one aptamer sequence is incorporated in the 3′ UTR of the NOI.

4. The expression vector of claim 1, wherein the at least one aptamer sequence comprises an oligonucleotide sequence of about 10 to about 200 base pairs.

5. The expression vector of claim 4, wherein the at least one aptamer sequence comprises an oligonucleotide sequence of about 20 to about 100 base pairs.

6. The expression vector of claim 5, wherein the at least one aptamer sequence comprises an oligonucleotide sequence of about 20 to about 60 base pairs.

7. The expression vector of claim 6, wherein the at least one aptamer sequence comprises an oligonucleotide sequence of about 20 to about 40 base pairs.

8. The expression vector of claim 1, wherein more than one copy of the at least one aptamer sequence are operably linked to the NOI.

9. The expression vector of claim 1, wherein at least two aptamer sequences are operably linked to the NOI, and wherein the aptamer sequences are different aptamer sequences.

10. The expression vector of claim 1, which is a viral vector.

11. The expression vector of claim 10, wherein the viral vector is selected from the group consisting of a retroviral vector, an adenoviral vector, an adeno-associated viral vector, a herpes viral vector, and a baculoviral vector.

12. The viral vector of claim 10, which is a lentiviral vector.

13. The expression vector of claim 1, further comprising a nucleotide sequence encoding an auxiliary protein, wherein the auxiliary protein and the at least one aptamer sequence form a complex that binds to a modulator.

14. The expression vector of claim 1, wherein the aptamer sequence is an aptazyme.

15. The expression vector of claim 1, wherein the NOI encodes glial-cell derived growth factor (GDNF).

16. The expression vector of claim 1, comprising at least two aptamer sequences linked by a spacer sequence.

17. An expression vector comprising at least one NOI, a first aptamer sequence and a second aptamer sequence linked by a spacer sequence, wherein the second aptamer sequence binds a modulator and wherein the second aptamer sequence and modulator form a complex that binds to the first aptamer sequence.

18. The expression vector of claim 17, wherein the NOI encodes GDNF.

19. The expression vector of claim 17, wherein the modulator is diflucan.

20. A method for regulating gene expression in a cell comprising the steps of:
a) providing an expression vector comprising at least one nucleotide sequence of interest (NOI) and at least one aptamer sequence, wherein the at least one aptamer sequence is operably linked to the at least one NOI;
b) providing a modulator capable of forming a complex with the at least one aptamer sequence;
c) administering the expression vector to a cell for the expression of the NOI; and
d) administering the modulator to the cell so that the modulator forms a complex with the at least one aptamer sequence, thereby regulating gene expression in the cell.

21. The method according to claim 20, wherein the at least one aptamer sequence is incorporated in the 5′ untranslated region (UTR) of the NOI.

22. The method according to claim 20, wherein the at least one aptamer sequence is incorporated in the 3′ UTR of the NOI.

23. The method according to claim 20, wherein the at least one aptamer sequence comprises an oligonucleotide sequence of about 10 to about 200 base pairs.

24. The method according to claim 23, wherein the at least one aptamer sequence comprises an oligonucleotide sequence of about 20 to about 100 base pairs.

25. The method according to claim 24, wherein the at least one aptamer sequence comprises an oligonucleotide sequence of about 20 to about 60 base pairs.

26. The method according to claim 25, wherein the at least one aptamer sequence comprises an oligonucleotide sequence of about 20 to about 40 base pairs.

27. The method according to claim 20, wherein more than one copy of the at least one aptamer sequence is operably linked to the NOI.

28. The method according to claim 20, wherein at least two of the aptamer sequences are different aptamer sequences.

29. The method according to claim 20, wherein the expression vector is a viral vector selected from the group consisting of retroviral vector, adenoviral vector, adeno-associated viral vector, herpes viral vector, and baculoviral vector.

30. The method according to claim 20, wherein the expression vector is a lentiviral vector.

31. The method according to claim 20, wherein the modulator is a small molecule having a dissociation constant (K_{d}) from at least about 10 μM to at least about 0.1 nM.

32. The method according to claim 20, wherein the cell is derived from the neuroepithelia and the modulator is capable of crossing the blood brain barrier.

33. The method according to claim 32, wherein the cell is selected from the group consisting of a neural cell and a neuroglial cell.

34. The method according to claim 20, further comprising, providing an auxiliary protein, wherein the modulator is further capable of forming a complex with the auxiliary protein, thereby regulating gene expression in the cell.

35. The method according to claim 34, wherein the auxiliary protein is selected from the group consisting of an endogenous protein and an exogenous protein.

36. The method according to claim 34, wherein the auxiliary protein is provided by administering an expression vector comprising a nucleotide sequence encoding the auxiliary protein.

37. The method according to claim 20, wherein the expression vector further comprises a nucleotide sequence encoding an auxiliary protein, wherein the auxiliary protein and the at least one aptamer sequence form a complex which binds to the modulator.

38. The method according to claim 20, wherein the aptamer sequence is an aptazyme.

39. A method for regulating gene expression in an animal comprising the steps of:
a) administering to the animal an expression vector comprising at least one nucleotide sequence of interest (NOI) and at least one aptamer sequence, wherein the at least one aptamer sequence is operably linked to the at least one NOI; and
b) administering a modulator to the animal cell so that the modulator forms a complex with the at least one aptamer sequence, thereby regulating gene expression in the animal.

40. The method according to claim 39, wherein the animal is a human.

41. The method according to claim 39, wherein the modulator is approved for animal or human use.

42. The method according to claim 39, wherein the NOI is GDNF.

43. The method according to claim 39, wherien the modulator is diflucan.
